SEQUENCE LISTING (27 sequences)

SEQ ID NO: 1 - protein, 20 aa, Artificial Sequence
  A molecular probe precursor
LSXQMEEEAVRLFIEWLXNG

SEQ ID NO: 2 - protein, 9 aa, Artificial Sequence
  Y1
HGEGTFTSD

SEQ ID NO: 3 - protein, 10 aa, Artificial Sequence
  Y2
GPSSGAPPPS

SEQ ID NO: 4 - protein, 39 aa, Artificial Sequence
  A molecular probe precursor
HGEGTFTSDLSXQMEEEAVRLFIEWLXNGGPSSGAPPPS

SEQ ID NO: 5 - protein, 31 aa, Artificial Sequence
  A molecular probe precursor
DLSXQMEEEAVRLFIEWLXNGGPSSGAPPPS

SEQ ID NO: 6 - protein, 20 aa, Artificial Sequence
  A radioactively labeled polypeptide
LSXQMEEEAVRLFIEWLXNG

SEQ ID NO: 7 - protein, 39 aa, Artificial Sequence; identical to SEQ ID NO: 4
  A radioactively labeled polypeptide

SEQ ID NO: 8 - protein, 31 aa, Artificial Sequence; identical to SEQ ID NO: 5
  A radioactively labeled polypeptide

SEQ ID NO: 9 - protein, 39 aa, Heloderma suspectum
  MOD_RES (39)..(39): AMIDATION
HGEGTFTSDLSKQMEEEAVRLFIEWLKNGGPSSGAPPPS

SEQ ID NO: 10 - protein, 31 aa, Heloderma suspectum
  MOD_RES (31)..(31): AMIDATION
DLSKQMEEEAVRLFIEWLKNGGPSSGAPPPS

SEQ ID NO: 11 - protein, 31 aa, Artificial Sequence
  A polypeptide used in a binding assay
DLSKQMEEEAVRLFIEWLRNGGPSSGAPPPS

SEQ ID NO: 12 - protein, 39 aa, Artificial Sequence
  A polypeptide used in a binding assay
HGEGTFTSDLSKQMEEEAVRLFIEWLRNGGPSSGAPPPS

SEQ ID NO: 13 - protein, 31 aa, Artificial Sequence; identical to SEQ ID NO: 11
  A protected peptide resin used in production of polypeptide used in a binding assay

SEQ ID NO: 14 - protein, 39 aa, Artificial Sequence; identical to SEQ ID NO: 12
  A protected peptide resin used in production of polypeptide used in a binding assay

SEQ ID NO: 15 - protein, 31 aa, Artificial Sequence; identical to SEQ ID NO: 11
  A polypeptide produced in Production Example 1

SEQ ID NO: 16 - protein, 31 aa, Artificial Sequence; identical to SEQ ID NO: 11
  A protected peptide resin produced in Production Example 1

SEQ ID NO: 17 - protein, 31 aa, Artificial Sequence; identical to SEQ ID NO: 11
  A molecular probe precursor produced in Production Example 1

SEQ ID NO: 18 - protein, 31 aa, Artificial Sequence; identical to SEQ ID NO: 10
  A molecular probe precursor used in Comparative Production Example 2

SEQ ID NO: 19 - protein, 31 aa, Artificial Sequence; identical to SEQ ID NO: 10
  A polypeptide produced in Comparative Production Example 1

SEQ ID NO: 20 - protein, 39 aa, Artificial Sequence; identical to SEQ ID NO: 12
  A polypeptide produced in Production Example 2

SEQ ID NO: 21 - protein, 39 aa, Artificial Sequence; identical to SEQ ID NO: 12
  A molecular probe precursor produced in Production Example 2

SEQ ID NO: 22 - protein, 39 aa, Artificial Sequence; identical to SEQ ID NO: 9
  A molecular probe precursor used in Comparative Production Example 2

SEQ ID NO: 23 - protein, 39 aa, Artificial Sequence; identical to SEQ ID NO: 9
  A polypeptide produced in Comparative Production Example 2

SEQ ID NO: 24 - protein, 39 aa, Artificial Sequence; identical to SEQ ID NO: 12
  A polypeptide produced in Example 3

SEQ ID NO: 25 - protein, 39 aa, Artificial Sequence
  A polypeptide produced in Production Example 3
HGEGTFTSDLSKQMEEEAVRLFIEWLXNGGPSSGAPPPS

SEQ ID NO: 26 - protein, 39 aa, Artificial Sequence; identical to SEQ ID NO: 25
  A molecular probe precursor used in Production Example 3

SEQ ID NO: 27 - protein, 39 aa, Artificial Sequence; identical to SEQ ID NO: 25
  A polypeptide produced in Production Example 4

(19) United States
(12) Patent Application Publication
(10) Pub. No.: US 2013/0052132 A1
(43) Pub. Date: Feb. 28, 2013

(54) Method for Producing Radioactively Labeled Polypeptide

(71) Applicants: Hideo Saji, Kyoto (JP); Nobuya Inagaki, Kyoto (JP); Hiroyuki Kimura, Kyoto (JP); Kentaro Toyoda, Kyoto (JP); Konomu Hirao, Kyoto (JP); Hirokazu Matsuda, Kyoto (JP)

(72) Inventors: Hideo Saji, Kyoto (JP); Nobuya Inagaki, Kyoto (JP); Hiroyuki Kimura, Kyoto (JP); Kentaro Toyoda, Kyoto (JP); Konomu Hirao, Kyoto (JP); Hirokazu Matsuda, Kyoto (JP)

(73) Assignees: ARKRAY, Inc., Kyoto (JP); Kyoto University, Kyoto (JP)

(21) Appl. No.: 13/369,903

(22) Filed: Feb. 9, 2012

Related U.S. Application Data
(60) Provisional application No. 61/441,062, filed on Feb. 9, 2011.
(60) Provisional application No. 61/522,973, filed on Aug. 12, 2011.

(30) Foreign Application Priority Data
Feb. 9, 2011 (JP) 2011-025963

(51) Int. Cl. C07K 14/00; A61K 51/08

(52) U.S. Cl. 424/1.69; 530/345; 530/300

(57) ABSTRACT

The present invention relates to a method for producing a polypeptide. The method includes labeling a molecular probe precursor represented by an amino acid sequence of the formula (1) using a labeling compound capable of labeling a lysine or lysine derivative:

(1)
Y₁-Leu-Ser-Xaa₁₂-Gln-Met-Glu-Glu-Glu-Ala-Val-Arg-
Leu-Phe-Ile-Glu-Trp-Leu-Xaa₂₇-Asn-Gly-Y₂

where Y₁ represents an amino acid sequence represented by the formula (2) or the amino acid sequence represented by the formula (2) with 1 to 8 amino acids from the N-terminus being deleted, Xaa₁₂ represents a lysine or lysine derivative, Xaa₂₇ represents a basic amino acid having no functional group at its side chain that reacts with the labeling compound or represents methyl lysine or acetylated lysine, and Y₂ represents an amino acid sequence represented by the formula (3) or the amino acid sequence represented by the formula (3) with 1 to 9 amino acids from the C-terminus being deleted:

(2)
His-Gly-Glu-Gly-Thr-Phe-Thr-Ser-Asp
(3)
Gly-Pro-Ser-Ser-Gly-Ala-Pro-Pro-Pro-Ser

### BACKGROUND OF INVENTION

1. Field of the Invention

The present invention relates to a method for producing a radioactively labeled polypeptide, the radioactively labeled polypeptide and a molecular probe precursor used in the production method.

2. Description of Related Art

The estimated number of type-II diabetics in Japan exceeded 8,800,000 according to the statistic in fiscal 2007, and the number has been growing year after year. Further, according to the International Diabetes Federation (IDF), the number of diabetics in the world has been growing in an analogous fashion. The estimated number of diabetics in the world is 285,000,000 as of the year 2010, and it is predicted that the number will reach 435,000,000 by the year 2030.

As a measure against this increase, interventions for preventing diabetes from developing have been made based on the glucose tolerance test, resulting, however, in unsatisfactory effects. The cause is as follows: at such a borderline stage that functional abnormalities are found by the glucose tolerance test, disorders of pancreatic islets have already advanced to a high degree, and this stage possibly is too late as a time for starting interventions.

Recently, it has been reported at home and overseas that even in type-II diabetics the amount of pancreatic islets already has decreased upon the development, and it has been considered that a further decrease in pancreatic β-cells after the development is one of the resistance factors against treatment for type-II diabetics. Therefore, if the amount of pancreatic islets and/or the amount of pancreatic β-cells can be detected, there is possibility for the clarification of pathogenesis, the ultra-early diagnosis, and the prevention of development of type-I and type-II diabetes. For this purpose, molecular probes that allow determination of the amount of pancreatic islets and/or the amount of pancreatic β-cells have been researched and developed.

In designing a molecular probe, various target molecules in pancreatic cells, particularly functional proteins specific in the β-cells, are being researched. Among these, GLP-1R (glucagon-like peptide-1 receptor) is being researched as a target molecule; GLP-1R is distributed in pancreatic β-cells, and is a seven-transmembrane G protein coupled receptor.

As molecular probes for imaging that use GLP-1R as a target molecule, the following are researched: a peptide derivative of GLP-1 having a C-terminus to which a labeling molecule is bonded; a peptide derivative of exendin-3 having a C-terminus to which a labeling molecule is bonded; and a peptide derivative of exendin-4 having a C-terminus to which a labeling molecule is bonded (e.g., JP 2008-511557 A; M. Gotthardt et al., “A new technique for in vivo imaging of specific GLP-1 binding sites”: First results in small rodents, Regulatory Peptides 137 (2006) 162-267; and M. Beche et al., “Are radiolabeled GLP-1 receptor antagonists useful for scintigraphy?”: 2009 SNM Annual Meeting, abstract, Oral Presentations No. 327). In addition, a derivative of exendin(9-39) is proposed (e.g., WO 2010/032509; M. Beche et al., “Are radiolabeled GLP-1 receptor antagonists useful for scintigraphy?”: 2009 SNM Annual Meeting, abstract, Oral Presentations No. 327; and H. Kimura et al., Development of in vivo imaging agents targeting glucagons-like peptide-1 receptor (GLP-1R) in pancreatic islets. 2009 SNM Annual Meeting, abstract, Oral Presentations No. 326).

### SUMMARY OF THE INVENTION

WO 2010/032509 discloses a method for obtaining a molecular probe. The method includes preparing, as a labeling precursor, an exendin(9-39) derivative in which protecting groups are bonded to amino groups other than those at labeling sites, and labeling and deprotecting the exendin(9-39) derivative. In this case, however, it is essential to perform deprotection after the labeling, which makes the procedure after the labeling complicated. Moreover, since it takes time from the labeling to obtaining the intended molecular probe, the radiochemical purity and radiochemical yields of the obtained molecular probe may decline.

On the other hand, attempts have been made to radioactively label exendin(9-39) without protective groups to amino groups other than those at labeling sites. In this case, however, it has been difficult to specifically label the target labeling sites. Furthermore, studies conducted by the present inventors have revealed the following: when radioactive labeling is performed using exendin(9-39) as it is, the lysine at position 19 is labeled preferentially, which makes it difficult to obtain a molecular probe in which the lysine at position 4 or the α-amino group at the N-terminus is labeled. The studies conducted by the present inventors have revealed similar results for exendin-4.

With the foregoing in mind, the present invention provides a method for producing, with high yield, exendin(9-39) and exendin-4 derivatives in which a target labeling site is radioactively labeled.

The present invention relates to a method for producing a radioactively labeled polypeptide. The method includes labeling a molecular probe precursor using a labeling compound capable of labeling an amino group of a lysine or lysine derivative. The molecular probe precursor is represented by an amino acid sequence of the formula (1) and a carboxylic group at the C-terminus of the molecular probe precursor is amidated:

(1)
(SEQ ID NO. 1)
Y₁-Leu-Ser-Xaa₁₂-Gln-Met-Glu-Glu-Glu-Ala-Val-Arg-Leu-Phe-Ile-Glu-Trp-
Leu-Xaa₂₇-Asn-Gly-Y₂

where Y₁ represents an amino acid sequence represented by the formula (2) or the amino acid sequence represented by the formula (2) with 1 to 8 amino acids from the N-terminus being deleted, Xaa₁₂ represents a lysine or lysine derivative, Xaa₂₇ represents a basic amino acid having no functional group at its side chain that reacts with the labeling compound, or represents methyl lysine or acetylated lysine, and Y₂ represents an amino acid sequence represented by the formula (3) or the amino acid sequence represented by the formula (3) with 1 to 9 amino acids from the C-terminus being deleted:

(2)
(SEQ ID NO. 2)
His-Gly-Glu-Gly-Thr-Phe-Thr-Ser-Asp
(3)
(SEQ ID NO. 3)
Gly-Pro-Ser-Ser-Gly-Ala-Pro-Pro-Pro-Ser.

With the present invention, exendin(9-39) and exendin-4 derivatives in which a target labeling site is radioactively labeled can be produced with high yields.

### BRIEF DESCRIPTION OF THE DRAWINGS

FIG. 1 is a graph showing exemplary results of a biodistribution experiment using a polypeptide of Example 1.

FIG. 2 shows images showing exemplary results of a two-dimensional imaging analysis using the polypeptide of Example 1.

FIG. 3 is a graph showing exemplary results of a biodistribution experiment using a polypeptide of Example 2.

FIG. 4 shows images showing exemplary results of a two-dimensional imaging analysis using the polypeptide of Example 2.

FIG. 5 shows images showing exemplary results of SPECT imaging using a polypeptide of Example 3.

FIG. 6 is a graph showing exemplary results of a biodistribution experiment using a polypeptide of Example 4.

FIG. 7 is a graph showing exemplary results of a biodistribution experiment using a polypeptide of Example 5.

### DETAILED DESCRIPTION OF THE EMBODIMENTS

The present invention is based on the findings that a radioactively labeled polypeptide in which a target labeling site is radiolabeled specifically can be produced easily in a short time with high yield by using the molecular probe precursor represented by the amino acid sequence of the formula (1) as a labeling precursor. Furthermore, the present invention is based on the findings that a radioactively labeled polypeptide that shows sufficient affinity to pancreatic β-cells and accumulates in pancreatic islets can be provided by using the molecular probe precursor.

In other words, the present invention relates to the following:

[1] A method for producing a radioactively labeled polypeptide, comprising

labeling a molecular probe precursor using a labeling compound capable of labeling an amino group of a lysine or lysine derivative,

wherein the molecular probe precursor is represented by an amino acid sequence of the formula (1) and a carboxylic group at the C-terminus of the molecular probe precursor is amidated:

(1)
(SEQ ID NO. 1)
Y₁-Leu-Ser-Xaa₁₂-Gln-Met-Glu-Glu-Glu-Ala-Val-Arg-Leu-Phe-Ile-Glu-Trp-
Leu-Xaa₂₇-Asn-Gly-Y₂

where Y₁ represents an amino acid sequence represented by the formula (2) or the amino acid sequence represented by the formula (2) with 1 to 8 amino acids from the N-terminus being deleted,

Xaa₁₂ represents a lysine or lysine derivative,

Xaa₂₇ represents a basic amino acid having no functional group at its side chain that reacts with the labeling compound, or represents methyl lysine or acetylated lysine, and

Y₂ represents an amino acid sequence represented by the formula (3) or the amino acid sequence represented by the formula (3) with 1 to 9 amino acids from the C-terminus being deleted:

(2)
(SEQ ID NO. 2)
His-Gly-Glu-Gly-Thr-Phe-Thr-Ser-Asp
(3)
(SEQ ID NO. 3)
Gly-Pro-Ser-Ser-Gly-Ala-Pro-Pro-Pro-Ser.

[2] The method according to [1], wherein the labeling compound is a compound represented by the formula (I)

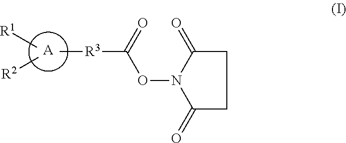

where Ar represents an aromatic hydrocarbon group or aromatic heterocyclic group, R¹ represents a substituent containing a radionuclide, R² represents a hydrogen atom or one or more substituents different from the substituent represented by R¹, and R³ represents a bond, C₁-C₆ alkylene group or C₁-C₆ oxyalkylene group.

[3] The method according to [1] or [2], wherein Xaa₂₇ in the formula (1) represents one selected from the group consisting of arginine, monomethyl lysine, dimethyl lysine, monoacetylated lysine, norarginine, homoarginine and histidine.
[4] The method according to any one of [1] to [3], wherein the molecular probe precursor is represented by an amino acid sequence of the formula (4) or (5):

(4)
(SEQ ID NO. 4)
His-Gly-Glu-Gly-Thr-Phe-Thr-Ser-Asp-Leu-Ser-Xaa₁₂-Gln-Met-Glu-Glu-Glu-
Ala-Val-Arg-Leu-Phe-Ile-Glu-Trp-Leu-Xaa₂₇-Asn-Gly-Gly-Pro-Ser-Ser-Gly-
Ala-Pro-Pro-Pro-Ser
(5)
(SEQ ID NO. 5)
Asp-Leu-Ser-Xaa₁₂-Gln-Met-Glu-Glu-Glu-Ala-Val-Arg-Leu-Phe-Ile-Glu-
Trp-Leu-Xaa₂₇-Asn-Gly-Gly-Pro-Ser-Ser-Gly-Ala-Pro-Pro-Pro-Ser

where Xaa₁₂ represents a lysine or lysine derivative, and Xaa₂₇ represents a basic amino acid having no functional group at its side chain that reacts with the labeling compound, or represents methyl lysine or acetylated lysine.

[5] A polypeptide represented by an amino acid sequence of the formula (6), wherein a carboxylic group at the C-terminus of the polypeptide is amidated:

(6)
(SEQ ID NO. 6)
Y₁-Leu-Ser-Xbb₁₂-Gln-Met-Glu-Glu-Glu-Ala-Val-Arg-Leu-Phe-Ile-Glu-Trp-
Leu-Xaa₂₇-Asn-Gly-Y₂

where Y₁ represents an amino acid sequence represented by the formula (2) or the amino acid sequence represented by the formula (2) with 1 to 8 amino acids from the N-terminus being deleted,

Xbb₁₂ represents a radioactively labeled lysine or lysine derivative,

Xaa₂₇ represents a basic amino acid having no functional group at its side chain that reacts with a labeling compound capable of labeling an amino group of a lysine or lysine derivative, or represents methyl lysine or acetylated lysine, and

Y₂ represents an amino acid sequence represented by the formula (3) or the amino acid sequence represented by the formula (3) with 1 to 9 amino acids from the C-terminus being deleted:

(2)
(SEQ ID NO. 2)
His-Gly-Glu-Gly-Thr-Phe-Thr-Ser-Asp
(3)
(SEQ ID NO. 3)
Gly-Pro-Ser-Ser-Gly-Ala-Pro-Pro-Pro-Ser.

[6] The polypeptide according to [5], wherein the polypeptide is represented by an amino acid sequence of the formula (7) or (8):

(SEQ ID NO. 7)
His-Gly-Glu-Gly-Thr-Phe-Thr-Ser-Asp-Leu-Ser-Xbb₁₂-
Gln-Met-Glu-Glu-Glu-Ala-Val-Arg-Leu-Phe-Ile-Glu-
Trp-Leu-Xaa₂₇-Asn-Gly-Gly-Pro-Ser-Ser-Gly-Ala-Pro-
Pro-Pro-Ser (7)
(SEQ ID NO. 8)
Asp-Leu-Ser-Xbb₁₂-Gln-Met-Glu-Glu-Glu-Ala-Val-Arg-
Leu-Phe-Ile-Glu-Trp-Leu-Xaa₂₇-Asn-Gly-Gly-Pro-Ser-
Ser-Gly-Ala-Pro-Pro-Pro-Ser (8)

where Xbb₁₂ represents a radioactively labeled lysine or lysine derivative, and Xaa₂₇ represents a basic amino acid having no functional group at its side chain that reacts with the labeling compound, or represents methyl lysine or acetylated lysine.

[7] The polypeptide according to [5] or [6], wherein the polypeptide is obtained by the method according to any one of [1] to [4].
[8] A molecular probe precursor used in the method according to any one of [1] to [4],

wherein the molecular probe precursor is represented by an amino acid sequence of the formula (1) and a carboxylic group at the C-terminus of the molecular probe precursor is amidated:

(SEQ ID No. 1)
Y₁-Leu-Ser-Xaa₁₂-Gln-Met-Glu-Glu-Glu-Ala-Val-Arg-
Leu-Phe-Ile-Glu-Trp-Leu-Xaa₂₇-Asn-Gly-Y₂ (1)

where Y₁ represents an amino acid sequence represented by the formula (2) or the amino acid sequence represented by the formula (2) with 1 to 8 amino acids from the N-terminus being deleted,

Xaa₁₂ represents a lysine or lysine derivative,

Xaa₂₇ represents a basic amino acid having no functional group at its side chain that reacts with a labeling compound capable of labeling an amino group of a lysine or lysine derivative, or represents methyl lysine or acetylated lysine, and

Y₂ represents an amino acid sequence represented by the formula (3) or the amino acid sequence of the formula (3) with 1 to 9 amino acids from the C-terminus being deleted:

(SEQ ID NO. 2)
His-Gly-Glu-Gly-Thr-Phe-Thr-Ser-Asp (2)
(SEQ ID NO. 3)
Gly-Pro-Ser-Ser-Gly-Ala-Pro-Pro-Pro-Ser (3).

[9] A composition for imaging comprising the polypeptide according to any one of [5] to [7] or the molecular probe precursor according to [5].
[10] A kit comprising the polypeptide according to any one of [5] to [7] and/or the molecular probe precursor according to [8].
[11] A method for imaging pancreatic β-cells, comprising detecting a radioactivity signal of the polypeptide according to any one of [5] to [7] from an analyte to which the polypeptide has been administered.
[12] The method according to [11], further comprising reconfiguring the detected signal to convert the signal into an image, and displaying the image.
[13] A method for determining an amount of pancreatic islets, the method comprising:

detecting a radioactivity signal of the polypeptide according to any one of [5] to [7] from an analyte to which the polypeptide has been administered; and

calculating an amount of pancreatic islets from the detected signal of the polypeptide.

[14] The method according to [13], further comprising presenting the calculated amount of pancreatic islets.

With the present invention, a polypeptide in which a target labeling site is radiolabeled specifically can be produced with high yield and the time involved in the labeling can be reduced. Thus, the present invention can preferably provide a molecular probe useful for imaging at low production cost in an efficient manner. Furthermore, a radioactively labeled polypeptide obtained by the production method of the present invention accumulates specifically in pancreatic β-cells and has an excellent blood clearance, for example. Thus, it may provide ways for the clarification of pathogenesis, the ultra-early diagnosis, and/or the prevention of development of diseases such as type-I and type-II diabetes.

[Method for Producing Radioactively Labeled Polypeptide]

The method for producing a radioactively labeled polypeptide according to the present invention (hereinafter referred also to as “the production method of the present invention”) includes labeling a molecular probe precursor using a labeling compound.

[Molecular Probe Precursor]

The molecular probe precursor used in the production method of the present invention (hereinafter referred also to as “the molecular probe precursor of the present invention”) is represented by the amino acid sequence of the formula (1) and a carboxylic group at the C-terminus of the molecular probe precursor is amidated.

(SEQ ID NO. 1)
Y₁-Leu-Ser-Xaa₁₂-Gln-Met-Glu-Glu-Glu-Ala-Val-Arg-
Leu-Phe-Ile-Glu-Trp-Leu-Xaa₂₇-Asn-Gly-Y₂ (1)

In the formula (1), Xaa₂₇ represents a basic amino acid having no functional group at its side chain that reacts with the labeling compound, or represents methyl lysine or acetylated lysine. The term “basic amino acid” as used herein refers to an amino acid having a basic side chain. The labeling compound is a compound capable of labeling an amino group of a lysine or lysine derivative. For example, the labeling compound is a labeling compound that will be described later, and preferably is a compound represented by the formula (I), and more preferably is [¹⁸F]N-succinimidyl 4-fluorobenzoate ([¹⁸F]SFB) and [^{123/124/125/131}I]N-succinimidyl 3-iodobenzoate ([^{123/124/125/131}I]SIB). For example, the basic amino acid having no functional group at its side chain that reacts with the labeling compound preferably is a basic amino acid having no functional group at its side chain that reacts with [¹⁸F]SFB and/or [^{123/124/125/131}I]SIB and more preferably is a basic amino acid having no amino group that reacts with [¹⁸F]SFB and/or [^{123/124/125/131}I]SIB under the conditions where labeling is performed using the labeling compound. For example, Xaa₂₇ is preferably a basic amino acid having a guanidyl group at its side chain. Xaa₂₇ may be a natural or unnatural amino acid. Examples of Xaa₂₇ include arginine, monomethyl lysine, dimethyl lysine, monoacetylated lysine, norarginine, homoarginine and histidine. In terms of obtaining a polypeptide highly accumulative in pancreas, arginine, monomethyl lysine and homoarginine are preferred, and arginine and monomethyl lysine are more preferred.

In the formula (1), Xaa₁₂ represents a lysine or lysine derivative. The term “lysine derivative” as used herein refers to an amino acid having an amino group at its side chain that reacts with the labeling compound capable of labeling an amino group, and examples of the lysine derivative include: a lysine whose side chain is bonded to a linker; ornithine, diaminopropionic acid and diaminobutyric acid; and these amino acids with the side chain being bonded to a linker. A lysine whose side chain is bonded to a linker is preferred. The linker preferably has, for example, at least a chain structure and an amino group. Examples of the chain structure include an alkyl chain and a polyethylene glycol chain. A group represented by the formula (II) is more preferable as the linker. In particular, Xaa₁₂ preferably is a lysine or lysine in which an amino group at its side chain is bonded to the group represented by the formula (II). In terms of obtaining a radioactively labeled polypeptide having a high blood clearance, a lysine in which an amino group at its side chain is bonded to the group represented by the formula (II) is more preferable.

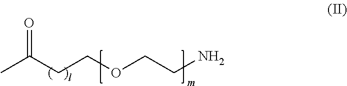

In the formula (II), l represents the number of methylene groups and m represents the number of oxyethylene groups. l is, for example, an integer of 0 to 8, preferably an integer of 0 to 5, more preferably an integer of 0 to 3, and still more preferably 0 or 1. m is an integer of 1 to 30. In terms of preventing the peptide derivative from accumulating in the liver, kidney, lungs and intestines and improving the pancreas/liver ratio and the pancreas/kidney ratio, m is preferably an integer of 3 to 30, more preferably an integer of 4 or more, 6 or more, 8 or more, 10 or more, or 12 or more. An upper limit to m is preferably an integer of 28 or less, 26 or less, 24 or less, 22 or less, 20 or less, 18 or less, 16 or less, 14 or less, or 12 or less. Further, in terms of improving the accumulation of the peptide derivative in the pancreas, m is, for example, an integer of 0 to 14, preferably an integer of 0 to 12, more preferably an integer of 2 to 8, and still more preferably 3, 4, 5, 6, 7 or 8.

Y₁ represents the amino acid sequence represented by the formula (2) or the amino acid sequence represented by the formula (2) with 1 to 8 amino acids from the N-terminus being deleted. In particular, the amino acid sequence represented by the formula (2) or Asp is preferred. In terms of obtaining a radioactively labeled polypeptide highly accumulative in pancreas and having a high blood clearance, the amino acid sequence represented by the formula (2) is more preferred. On the other hand, Asp is more preferred in terms of the production costs.

(SEQ ID NO. 2)
His-Gly-Glu-Gly-Thr-Phe-Thr-Ser-Asp (2)

Y₂ represents the amino acid sequence represented by the formula (3) or the amino acid sequence represented by the formula (3) with 1 to 9 amino acids from the C-terminus being deleted. Y₂ preferably represents the amino acid sequence represented by the formula (3).

(SEQ ID NO. 3)
Gly-Pro-Ser-Ser-Gly-Ala-Pro-Pro-Pro-Ser (3)

The molecular probe precursor is preferably represented by the amino acid sequence of the formula (4) or (5). In terms of obtaining a radioactively labeled polypeptide highly accumulative in pancreas and having a high blood clearance, the amino acid sequence represented by the formula (4) is more preferred. On the other hand, the amino acid sequence represented by the formula (5) is more preferred in terms of the production costs. In the formulae (4) and (5), Xaa₁₂ and Xaa₂₇ are as described above.

(SEQ ID NO. 4)
His-Gly-Glu-Gly-Thr-Phe-Thr-Ser-Asp-Leu-Ser-Xaa₁₂-
Gln-Met-Glu-Glu-Glu-Ala-Val-Arg-Leu-Phe-Ile-Glu-
Trp-Leu-Xaa₂₇-Asn-Gly-Gly-Pro-Ser-Ser-Gly-Ala-Pro-
Pro-Pro-Ser (4)
(SEQ ID NO. 5)
Asp-Leu-Ser-Xaa₁₂-Gln-Met-Glu-Glu-Glu-Ala-Val-Arg-
Leu-Phe-Ile-Glu-Trp-Leu-Xaa₂₇-Asn-Gly-Gly-Pro-Ser-
Ser-Gly-Ala-Pro-Pro-Pro-Ser (5)

The α-amino group at the N-terminus of the molecular probe precursor may be protected by a protecting group, modified with a modifying group having no electric charge or not modified. In terms of canceling the positive electric charge of the α-amino group at the N-terminus to prevent the radioactively labeled polypeptide from accumulating in the kidney, allowing more selective labeling and further simplifying the operations after the labeling so as to reduce the production time, the α-amino group at the N-terminus may be modified with a modifying group having no electric charge.

Examples of the modifying group having no electric charge include 9-fluorenylmethyloxycarbonyl group (Fmoc), tert-butoxycarbonyl group (Boc), benzyloxycarbonyl group (Cbz), 2,2,2-trichloroethoxycarbonyl group (Troc), allyloxycarbonyl group (Alloc), 4-methoxytrityl group (Mmt), amino group, alkyl groups having 3 to 20 carbon atoms, 9-fluoreneacetyl group, 1-fluorenecarboxylic acid group, 9-fluorenecarboxylic acid group, 9-fluorenone-1-carboxylic acid group, benzyloxycarbonyl group, xanthyl group (Xan), trityl group (Trt), 4-methyltrityl group (Mtt), 4-methoxy-2,3,6-trimethyl-benzenesulfonyl group (Mtr), mesitylene-2-sulfonyl group (Mts), 4,4-dimethoxybenzohydryl group (Mbh), tosyl group (Tos), 2,2,5,7,8-pentamethylchroman-6-sulfonyl group (Pmc), 4-methylbenzyl group (MeBzl), 4-methoxybenzyl group (MeOBzl), benzyloxy group (BzlO), benzyl group (Bzl), benzoyl group (Bz), 3-nitro-2-pyridinesulfenyl group (Npys), 1-(4,4-dimethyl-2,6-diaxocyclohexylidene)ethyl group (Dde), 2,6-dichlorobenzyl group (2,6-DiCl-Bzl), 2-chlorobenzyloxycarbonyl group (2-Cl—Z), 2-bromobenzyloxycarbonyl group (2-Br—Z), benzyloxymethyl group (Bom), cyclohexyloxy group (cHxO), t-butoxymethyl group (Bum), t-butoxy group (tBuO), t-butyl group (tBu), acetyl group (Ac), trifluoroacetyl group (TFA), o-bromobenzyloxycarbonyl group, t-butyldimethylsilyl group, 2-chlorobenzyl (Cl-z) group, cyclohexyl group, cyclopentyl group, isopropyl group, pivalyl group, tetrahydropyran-2-yl group, and trimethylsilyl group. Among these, preferably, the modifying group is acetyl group, benzyl group, benzyloxymethyl group, o-bromobenzyloxycarbonyl group, t-butyl group, t-butyldimethylsilyl group, 2-chlorobenzyl group, 2,6-dichlorobenzyl group, cyclohexyl group, cyclopentyl group, isopropyl group, pivaloyl group, tetrahydropyran-2-yl group, tosyl group, trimethylsilyl group, or trityl group. More preferably, the modifying group is acetyl group.

For the protecting group, it is possible to use any known protecting groups capable of protecting the α-amino group at the N-terminus of the molecular probe precursor during the labeling. The protecting group is not particularly limited, and examples of the same include Fmoc, Boc, Cbz, Troc, Alloc, Mmt, alkyl groups having 3 to 20 carbon atoms, 9-fluoreneacetyl group, 1-fluorenecarboxylic acid group, 9-fluorenecarboxylic acid group, 9-fluorenone-1-carboxylic acid group, benzyloxycarbonyl group, Xan, Trt, Mtt, Mtr, Mts, Mbh, Tos, Pmc, MeBzl, MeOBzl, BzlO, Bzl, Bz, Npys, Dde, 2,6-DiCl-Bzl, 2-Cl—Z, 2-Br—Z, Bom, cHxO, Bum, tBuO, tBu, Ac and TFA. Fmoc or Boc are preferred in terms of the handing.

The molecular probe precursor of the present invention may include a polypeptide having homology with the polypeptide consisting of the amino acid sequence represented by the formula (1), (4) or (5) and bondable to GLP-1R of pancreatic β-cells after being labeled. Examples of the polypeptide having homology with the polypeptide consisting of the amino acid sequence represented by the formula (1), (4) or (5) include polypeptides that are the same as the polypeptide consisting of the amino acid sequence represented by the formula (1), (4) or (5) with one to several amino acids being deleted, added or replaced and polypeptides 80% or more homologous with the amino acid sequence of the polypeptide.

The molecular probe precursor of the present invention may be in the form of a solution, powder or the like. The molecular probe precursor preferably is in the form of a powder, and more preferably is in the form of a freeze-dried powder (freeze-dried formulation) in terms of the handling.

The molecular probe precursor of the present invention can be produced by peptide synthesis in accordance with a typical method such as the Fmoc method, and the peptide synthesis method is not particularly limited.

[Labeling Compound]

The labeling compound is not particularly limited as long as it is a labeling compound capable of labeling an amino group of a lysine or lysine derivative, for example, a labeling compound having a radionuclide and capable of introducing a radioactive labeling group by reacting with the amino group of the lysine or lysine derivative. A labeling compound that reacts with the amino group at the side chain of a lysine or lysine derivative and bonds a radioactive labeling group to the amino group is preferred. With the production method of the present invention, the lysine or lysine derivative represented by Xaa₁₂ can be labeled specifically because the above-described molecular probe precursor is used.

Examples of the radionuclide include ¹¹C, ¹³N, ¹⁵O, ¹⁸F, ⁶⁴Cu, ⁶⁷Ga, ⁶⁸Ga, ⁷⁵Br, ⁷⁶Br, ⁷⁷Br, ⁸²Rb, ^{99m}Tc, ¹¹¹In, ¹²³I, ¹²⁴I, ¹²⁵I, ¹³¹I, and ¹⁸⁶Re. In terms of performing positron emission tomography (PET), the radionuclide preferably is a positron emission nuclide such as ¹¹C, ¹³N, ¹⁵O, ¹⁸F, ⁶²Cu, ⁶⁴Cu, ⁶⁸Ga, ⁷⁵Br, ⁷⁶Br, ⁸²Rb, or ¹²⁴I. In terms of performing single photon emission computed tomography (SPECT), the radionuclide preferably is a γ-emission nuclide such as ⁶⁷Ga, ^{99m}Tc, ⁷⁷Br, ¹¹¹In, ¹²³I, or ¹²⁵I, and more preferably is ⁷⁷Br, ^{99m}Tc, ¹¹¹In, ¹²³I, or ¹²⁵I. Among these, radioactive halogen nuclides such as ¹⁸F, ⁷⁵Br, ⁷⁶Br, ⁷⁷Br, ¹²³I, and ¹²¹I are preferred more, and ¹⁸F, ¹²³I, and ¹²¹I are preferred particularly as the radionuclide.

In terms of obtaining a radioactively labeled polypeptide highly accumulative in pancreas and having a high blood clearance, it is preferable that the labeling compound has a group represented by the formula (III), it is more preferable that the labeling compound is a succinimidyl ester compound in which the group represented by the formula (III) is bonded to succinimide through ester bonding, and it is still more preferable that the labeling compound is a compound represented by the formula (I).

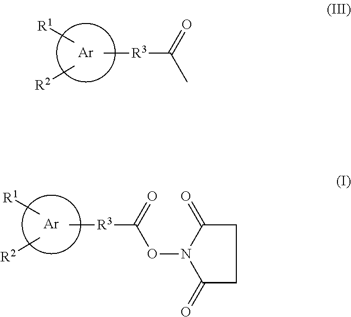

In the formulae (I) and (III), Ar represents an aromatic hydrocarbon group or aromatic heterocyclic group. The aromatic hydrocarbon group preferably is an aromatic hydrocarbon group having 6 to 18 carbon atoms, and examples of the same include phenyl group, o-tolyl group, m-tolyl group, p-tolyl group, 2,4-xylyl group, p-cumenyl group, mesityl group, 1-naphthyl group, 2-naphthyl group, 1-anthryl group, 2-anthryl group, 9-anthryl group, 1-phenanthryl group, 9-phenanthryl group, 1-acenaphthyl group, 2-azulenyl group, 1-pyrenyl group, 2-triphenylenyl group, o-biphenylyl group, m-biphenylyl group, p-biphenylyl group, and terphenyl group. The aromatic heterocyclic group preferably is a 5 to 10-membered heterocyclic group having one or two nitrogen atoms, oxygen atoms, or sulfur atoms, and examples of the same include triazolyl group, 3-oxadiazolyl group, 2-furyl group, 3-furyl group, 2-thienyl group, 3-thienyl group, 1-pyrrolyl group, 2-pyrrolyl group, 3-pyrrolyl group, 2-pyridyl group, 3-pyridyl group, 4-pyridyl group, 2-pyradyl group, 2-oxazolyl group, 3-isoxyazolyl group, 2-thiazolyl group, 3-isothiazolyl group, 2-imidazolyl group, 3-pyrazolyl group, 2-quinolyl group, 3-quinolyl group, 4-quinolyl group, 5-quinolyl group, 6-quinolyl group, 7-quinolyl group, 8-quinolyl group, 1-isoquinolyl group, 2-quinoxalynyl group, 2-benzofuryl group, 2-benzothienyl group, N-indolyl group, and N-carbazolyl group. Ar preferably is, among these, phenyl group, triazolyl group, or pyridyl group, and more preferably is phenyl group.

In the formulae (I) and (III), R¹ represents a substituent containing a radionuclide, and examples of the same include radionuclides, radionuclide-substituted C₁-C₃ alkyl groups, radionuclide-substituted C₁-C₃ alkoxy groups. Although examples of the radionuclide are as described above, a radioactive halogen nuclide is preferable among the examples described above.

The term “C₁-C₃ alkyl group” as used herein refers to an alkyl group having 1 to 3 carbon atoms, and examples of the same include methyl group, ethyl group, and propyl group. The term “radionuclide-substituted C₁-C₃ alkyl group” as used herein refers to an alkyl group having 1 to 3 carbon atoms in which a radionuclide substitutes for a hydrogen atom. The term “C₁-C₃ alkoxy group” as used herein refers to an alkoxy group having 1 to 3 carbon atoms, and examples of the same include methoxy group, ethoxy group, and propoxy group. The term “radionuclide-substituted C₁-C₃ alkoxy group” as used herein refers to an alkoxy group having 1 to 3 carbon atoms in which a radionuclide substitutes for a hydrogen atom.

R¹ preferably is, for example, a substituent containing ¹⁸F, ^{75/76/77}Br, or ^{123/124/125/131}I. In terms of performing PET, R¹ preferably is a substituent containing ¹⁸F, ⁷⁵Br, ⁷⁶Br, or ¹²⁴I, and more preferably is ¹⁸F, ⁷⁵Br, ⁷⁶Br, or ¹²⁴I. In terms of performing SPECT, R¹ preferably is a substituent containing ⁷⁷Br, ¹²³I, or ¹²⁵I, and more preferably is ⁷⁷Br, ¹²³I, or ¹²⁵I. It should be noted that the term “^{75/76/77}Br” as used herein refers to ⁷⁵Br, ⁷⁶Br or ⁷⁷Br and the term “^{123/124/125/131}I” refers to ¹²³I, ¹²⁴I, ¹²⁵I, or ¹³¹I.

In the formulae (I) and (III), R² represents a hydrogen atom or one or more substituents different from the substituent represented by R¹. Although R² may be a hydrogen atom or one or more substituents, a hydrogen atom is preferred. That is, in the formula (I) or (III), Ar preferably is not replaced with a substituent other than the substituent represented by R¹. When R² represents two or more substituents, the substituents may be the same or they may be different from each other. Examples of the substituent include hydroxyl group, electron withdrawing groups, electron donative groups, C₁-C₆ alkyl groups, C₂-C₆ alkenyl groups, and C₂-C₆ alkynyl groups. Examples of electron withdrawing groups include cyano group, nitro group, halogen atoms, carbonyl group, sulfonyl group, acetyl group, and phenyl group. Examples of halogen atoms include fluorine atom, chlorine atom, bromine atom, and iodine atom. The term “C₁-C₆ alkyl group” as used herein refers to an alkyl group having 1 to 6 carbon atoms, and examples of the same include methyl group, ethyl group, propyl group, isopropyl group, butyl group, isobutyl group, sec-butyl group, tert-butyl group, pentyl group, isopentyl group, and hexyl group. The term “C₂-C₆ alkenyl group” as used herein refers to an alkenyl group having 2 to 6 carbon atoms, and examples of the same include vinyl group, 1-propenyl group, 2-propenyl group, isopropenyl group, 1-butenyl group, 2-butenyl group, and 3-butenyl group. The term “C₂-C₆ alkynyl group” as used herein refers to an alkynyl group having 2 to 6 carbon atoms, and examples of the same include ethynyl group, 1-propynyl group, 2-propynyl group, 1-butynyl group, 2-butynyl group, and 3-butynyl group. Among these, the substituent preferably is a hydroxyl group or electron attractive group.

R³ preferably represents a bond, alkylene group having 1 to 6 carbon atoms, or oxyalkylene group having 1 to 6 carbon atoms. Examples of the alkylene group having 1 to 6 carbon atoms include straight-chain or branched-chain alkylene groups such as methylene group, ethylene group, propylene group, butylene group, pentyl group, and hexyl group. Examples of the oxyalkylene group having 1 to 6 carbon atoms include oxymethylene group, oxyethylene group, oxypropylene group, oxybutylene group, and oxypentyl group. R³ preferably is a bond, methylene group, or ethylene group, and more preferably is a bond in terms of obtaining a radioactively labeled polypeptide highly accumulative in pancreas and having a high blood clearance. Further, when R¹ is a substituent containing ¹⁸F, and preferably is ¹⁸F, R³ preferably is a bond. When R¹ is a substituent containing ^{123/124/125/131}I, and preferably is ^{123/124/125/131}I, R³ preferably is a methylene group, ethylene group or bond, and more preferably is a bond.

The labeling compound preferably is a compound represented by the formula (Ia), among others. In the formula (Ia), R¹ may be at the ortho-, para- or meta-position relative to R³. When R¹ is ¹⁸F, R¹ preferably is at the para-position (4-position) relative to R³, and when R¹ is ^{123/124/125/131}I, R¹ preferably is at the meta-position (3- or 5-position) relative to R³. The labeling compound more preferably is a compound represented by the formula (Ib) (i.e., [¹⁸F]SFB), compound represented by the formula (Ic) (i.e., [^{123/124/125/131}I]SIB) or compound represented by the formula (Id). The compound represented by the formula (Ic) or (Ib) is more preferred in terms of obtaining a radioactively labeled polypeptide highly accumulative in pancreas and having a high blood clearance. The compound represented by the formula (Id) is more preferred in terms of its general versatility.

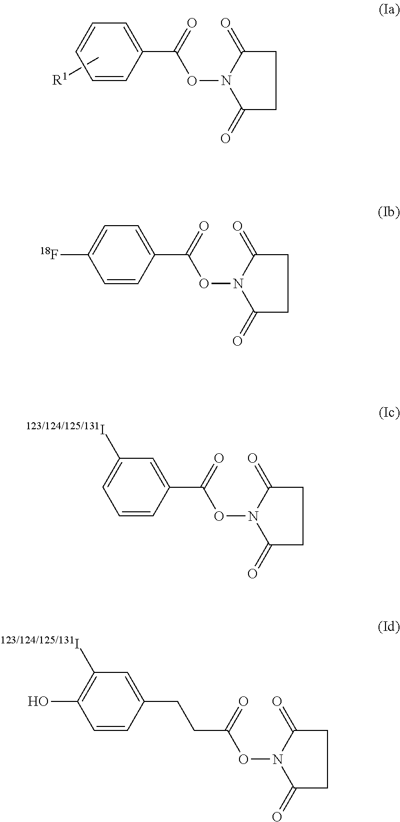

Labeling using the labeling compound can be performed by a known method. For example, labeling can be performed by adding a solution containing the molecular probe precursor to the labeling compound, followed by allowing the labeling compound and the molecular probe precursor to react with each other while adjusting the pH to be within a certain range.

In order to remove protecting groups bonded to the labeled polypeptide, the production method of the present invention may include deprotecting the radioactively labeled polypeptide. The deprotection of the polypeptide can be performed by a known method in accordance with the type of the protecting groups.

In terms of producing a radioactively labeled polypeptide of high purity, the production method of the present invention may further include purifying the labeled polypeptide. For the purification, any known separation operation for purifying peptide or protein can be used, for example. Examples of separation operations include ion-exchange chromatography, hydrophobic chromatography, reversed-phase chromatography, and high-performance liquid chromatography (HPLC), and these may be used in combination as needed.

The production method of the present invention may include synthesizing the labeling compound and/or synthesizing the molecular probe precursor. In this case, the synthesis of the labeling compound and the labeling of the molecular probe precursor may be performed using a single automatic synthesizing device or the synthesis of the molecular probe precursor, the synthesis of the labeling compound, and the labeling of the molecular probe precursor may be performed using a single automatic synthesizing device.

[Radioactively Labeled Polypeptide]

Another aspect of the present invention relates to a polypeptide represented by an amino acid sequence of the formula (6), wherein a carboxylic group at the C-terminus of the polypeptide is amidated (hereinafter referred also to as “the polypeptide of the present invention”). With the polypeptide of the present invention, it is possible to provide a molecular probe useful for imaging, which accumulates specifically in pancreatic β-cells and has an excellent blood clearance. The polypeptide of the present invention can be produced by the production method of the present invention described above.

(SEQ ID NO. 6)
Y₁-Leu-Ser-Xbb₁₂-Gln-Met-Glu-Glu-Glu-Ala-Val-Arg-
Leu-Phe-Ile-Glu-Trp-Leu-Xaa₂₇-Asn-Gly-Y₂ (6)

In the formula (6), Y₁, Xaa₂₇, and Y₂ are as described above in connection with the formula (1).

In the formula (6), Xbb₁₂ represents a radioactively labeled lysine or lysine derivative. Examples of the radioactively labeled lysine include a lysine in which an amino group at its side chain is bonded to a radioactive labeling group. Examples of the radioactive labeling group include those described later. Examples of the radioactively labeled lysine derivative include a lysine whose side chain is bonded to a linker having a radioactive labeling group, radioactively labeled ornithine, diaminopropionic acid and diaminobutyric acid, and these amino acids with the side chain being bonded to a linker having a radioactive labeling group. A lysine whose side chain is bonded to a linker having a radioactive labeling group is preferred. The linker having a radioactive labeling group preferably has, for example, at least a chain structure and a radioactive labeling group, and a linker in which a chain structure and a radioactive labeling group are bonded to each other through an amino group is more preferred. Examples of the chain structure include an alkyl chain and a polyethylene glycol chain. The linker in which an amino group at its side chain has a radioactive labeling group preferably is a group represented by the formula (IV). In particular, Xbb₁₂ preferably is a lysine in which an amino group at its side chain is radioactively labeled or lysine in which an amino group at its side chain is bonded to the group represented by the formula (IV). In terms of obtaining a radioactively labeled polypeptide having a high blood clearance, Xbb₁₂ more preferably is a lysine in which an amino group at its side chain is bonded to the group represented by the formula (IV).

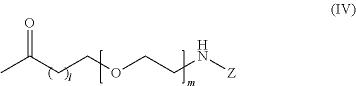

In the formula (IV), l and m are as described above in connection with the formula (II). Z represents a radioactive labeling group. Any of various known labeling groups can be used as the radioactive labeling group. In terms of obtaining a radioactively labeled polypeptide highly accumulative in pancreas and having a high blood clearance, the radioactive labeling group preferably is a group represented by the formula (V), more preferably is a group represented by the formula (Va), even more preferably is a group represented by the formula (Vb), (Vc) or (Vd), and still more preferably is a group represented by the formula (Vb) or (Vc). In the formula (V), Ar, R¹, R² and R³ are as described above in connection with the formula (I). In the formula (Va), R¹ is as described above in connection with the formula (I).

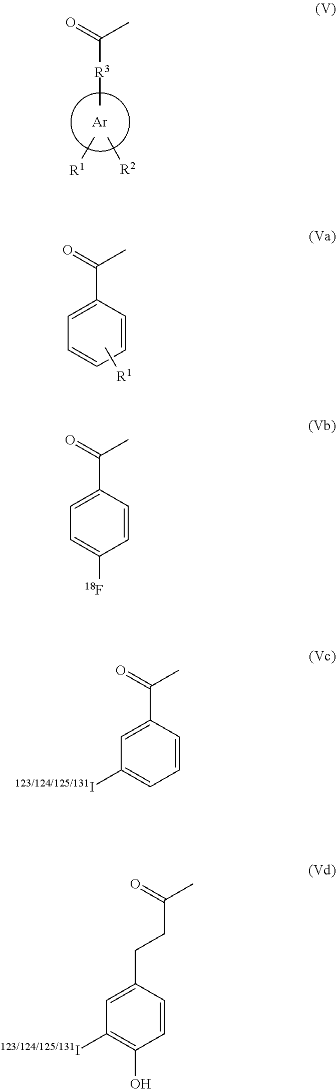

In the polypeptide of the present invention, the radioactive labeling group may contain a radioactive metal nuclide and a chelating site that can chelate the radioactive metal nuclide in terms of performing labeling with a metal radioisotope (radioactive metal nuclide). Examples of compounds that may form a chelating site include diethylenetriaminepentaacetic acid (DTPA), 6-hydrazinopyridine-3-carboxylic acid (HYNIC), tetraazacyclododecanetetraacetic acid (DOTA), dithisosemicarbazone (DTS), diaminedithiol (DADT), mercaptoacetylglycylglycylglycine (MAG3), monoamidemonoaminedithiol (MANIA), diamidedithiol (DADS), and propylene diamine dioxime (PnAO).

The α-amino group at the N-terminus of the polypeptide of the present invention may not be modified or may be modified with a modifying group having no electric charge. Examples of the modifying group having no electric charge are the same as those described above in connection with the molecular probe precursor of the present invention.

The polypeptide of the present invention preferably is represented by an amino acid sequence of the formula (7) or (8). In terms of obtaining a polypeptide highly accumulative in pancreas and having a high blood clearance, the amino acid sequence represented by the formula (7) is more preferred. In terms of the production costs, the amino acid sequence represented by the formula (8) is more preferred. In the formulae (7) and (8), Xbb₁₂ and Xaa₂₇ are as describe above.

(SEQ ID No. 7)
His-Gly-Glu-Gly-Thr-Phe-Thr-Ser-Asp-Leu-Ser-Xbb₁₂-
Gln-Met-Glu-Glu-Glu-Ala-Val-Arg-Leu-Phe-Ile-Glu-
Trp-Leu-Xaa₂₇-Asn-Gly-Gly-Pro-Ser-Ser-Gly-Ala-Pro-
Pro-Pro-Ser (7)
(SEQ ID No. 8)
Asp-Leu-Ser-Xbb₁₂-Gln-Met-Glu-Glu-Glu-Ala-Val-Arg-
Leu-Phe-Ile-Glu-Trp-Leu-Xaa₂₇-Asn-Gly-Gly-Pro-Ser-
Ser-Gly-Ala-Pro-Pro-Pro-Ser (8)

The polypeptide of the present invention may include a polypeptide having homology with the polypeptide consisting of the amino acid sequence represented by the formula (6), (7) or (8) and bondable to GLP-1R of pancreatic β-cells. Examples of the polypeptide having homology with the polypeptide consisting of the amino acid sequence represented by the formula (6), (7) or (8) may include polypeptides that are the same as the polypeptide consisting of the amino acid sequence represented by the formula (6), (7) or (8) with one to several amino acids being deleted, added or replaced and polypeptides 80% or more homologous with the amino acid sequence of the polypeptide.

For example, the polypeptide of the present invention can be used for imaging of pancreatic islets, preferably for imaging of pancreatic β-cells, and more preferably for a molecular probe for imaging GLP-1R of pancreatic β-cells. Further, the polypeptide of the present invention can be used for imaging for prevention, treatment, or diagnosis of diabetes, for example. Further, the polypeptide of the present invention can be used for, for example, a composition, an imaging reagent, a contrast medium and an diagnostic imaging agent that contain the polypeptide of the present invention as an active ingredient and are used for the various kinds of imaging as described above. Although the composition, the diagnostic imaging agent and the like may be in the form of a solution or powder, for example, they preferably are in the form of a solution, and more preferably are in the form of a parenteral solution in consideration of the half-life and radioactive decay of the radionuclide.

[Composition for Imaging]

Still another aspect of the present invention relates to a composition for imaging containing the polypeptide of the present invention or the molecular probe precursor of the present invention. The composition for imaging of the present invention may be in the form of a solution, powder or the like, for example. When the composition for imaging of the present invention contains the polypeptide of the present invention, the composition for imaging preferably is in the form of a solution, and more preferably is in the form of a parenteral solution in consideration of the half-life and radioactive decay of the radionuclide. When the composition for imaging of the present invention contains the molecular probe precursor of the present invention, the composition for imaging preferably is in the form of a powder, and more preferably is in the form of a freeze-dried powder (freeze-dried formulation) in terms of the handling.

The composition for imaging of the present invention may contain medicinal additives such as a carrier. The term medicinal additive as used herein refers to a compound that has been approved as a medicinal additive in the Japanese, US and/or European pharmacopoeia. Aqueous solvents and non-aqueous solvents can be used as the carrier. Examples of aqueous solvents include a potassium phosphate buffer solution, a physiological saline solution, a Ringer's solution, and distilled water. Examples of non-aqueous solvents include polyethylene glycol, vegetable oils, ethanol, glycerin, dimethylsulfoxide and propylene glycol.

[Kit]

Still another aspect of the present invention relates to a kit including the polypeptide of the present invention and/or the molecular probe precursor of the present invention. Examples of embodiments of the kit include a kit for producing the polypeptide of the present invention, a kit for imaging pancreatic β-cells, a kit for imaging GLP-1R of pancreatic β-cells, a kit for determining an amount of pancreatic islets, and a kit for prevention, treatment, or diagnosis of diabetes. Preferably, in each of these embodiments, the kit of the present invention includes an instruction manual suited to each embodiment. The instruction manual may be packed with the kit or may be provided on the web.

The form of the polypeptide of the present invention is not particularly limited, and the polypeptide may be in the form of a solution, powder or the like, for example. In consideration of the half-life and radioactive decay of the radionuclide, the polypeptide of the present invention preferably is in the form of a solution, and more preferably is in the form of a parenteral solution. The form of the molecular probe precursor of the present invention is not particularly limited, and the molecular probe precursor may be in the form of a solution, powder or the like, for example. In terms of the handling, the molecular probe precursor of the present invention preferably is in the form of a powder, and more preferably is in the form of a freeze-dried powder (freeze-dried formulation).

When the kit of the present invention includes the molecular probe precursor, the kit may include a labeling compound used for labeling the molecular probe precursor, compounds as starting materials of the labeling compound and other reagents used for radioactive labeling. The labeling compound is as described above.

Examples of the starting materials include starting materials of the labeling compound represented by the formula (Ib) and those of the labeling compound represented by the formula (Ic). Examples of the starting materials of the labeling compound represented by the formula (Ib) include ester derivatives of 4-(trimethylammonium triflate)benzoic acid. Examples of ester derivatives of 4-(trimethylammonium triflate)benzoic acid include methyl ester, ethyl ester, t-butyl ester and pentamethyl ester. Examples of other starting materials of the labeling compound represented by the formula (Ib) include ethyl 4-(trimethylammonium triflate)benzoate, ethyl 4-(tosyloxy)benzoate, and ethyl 4-(methylsulfonyloxy)benzoate. Examples of starting materials of the labeling compound represented by the formula (Ic) include 2,5-dioxopyrrolidin-1-yl 3-(tributylstannyl)benzoate, 2,5-dioxopyrrolidin-1-yl 3-bromobenzoate, 2,5-dioxopyrrolidin-1-yl 3-chlorobenzoate and 2,5-dioxopyrrolidin-1-yl 3-iodobenzoate. Examples of the other reagents used for radioactive labeling include reagents containing a radionuclide used for synthesizing the labeling compound.

The kit of the present invention further may include a container for containing the polypeptide of the present invention and/or the molecular probe precursor of the present invention. Examples of the container include a syringe and a vial.

The kit of the present invention may further include components for preparing a molecular probe, such as a buffer and an osmotic regulator, and an instrument used for administering the polypeptide, such as a syringe.

The kit including the molecular probe precursor may include, for example, an automatic synthesizing device for synthesizing the labeling compound. In addition to synthesizing the labeling compound, the automatic synthesizing device may also be capable of labeling the molecular probe precursor using the synthesized labeling compound, deprotecting the labeled polypeptide, and synthesizing the molecular probe precursor.

[Method for Imaging]

Still another aspect of the present invention relates to a method for imaging pancreatic β-cells. The method includes detecting a radioactivity signal of the polypeptide of the present invention from an analyte to which the polypeptide has been administered. With the method for imaging of the present invention, imaging of pancreatic β-cells, preferably imaging of GLP-1R of pancreatic β-cells can be performed because the polypeptide of the present invention is used. Examples of the analyte include humans and/or mammals other than humans.

The method for imaging of the present invention includes, as a first embodiment, detecting a radioactivity signal of the polypeptide of the present invention from an analyte to which the polypeptide has been administered in advance. For example, the signal preferably is detected after time sufficient for detecting the signal has elapsed from the administration of the polypeptide.

The method for imaging of the present invention may include, for example, reconfiguring the detected signal to convert the signal into an image and displaying the image, and/or converting the detected signal into numbers and presenting an accumulation amount. Displaying includes, for example, displaying the image on a monitor and printing the same. Presenting includes, for example, storing the calculated accumulation amount and outputting the same to the outside.

The detection of the signal can be determined appropriately in accordance with the type of the radionuclide of the polypeptide to be used, and PET, SPECT and the like can be used to detect the signal. SPECT includes, for example, determining, with use of a gamma camera, γ-rays emitted from an analyte to which the polypeptide of the present invention has been administered. The determination with the use of a gamma camera includes, for example, measuring radiation (γ-rays) emitted from the radionuclide of the polypeptide during a certain time unit, and preferably includes determining the direction in which the radiation is emitted and the radiation dose during a certain time unit. The method for imaging of the present invention further may include presenting, as a cross-sectional image, the determined distribution of the polypeptide obtained by the measurement of the radiation, and reconfiguring the obtained cross-sectional image.

PET includes, for example, simultaneously measuring γ-rays generated upon annihilation of positrons with electrons, with use of a detector for PET, from an analyte to which the polypeptide has been administered, and further may include figuring a three-dimensional distribution of positions of positron-emitting radionuclides, based on the measurement results.

The determination by means of X-ray CT and/or MRI may be performed, together with the determination by means of SPECT or PET. This makes it possible to obtain, for example, a fusion image obtained by fusion of an image obtained by SPECT or PET (functional image) with an image obtained by CT or MRI (morphological image).

The method for imaging of the present invention includes, as a second embodiment, administering the polypeptide of the present invention to an analyte and detecting a radioactivity signal of the polypeptide from the analyte to which the polypeptide has been administered. The detection of the signal and the reconfiguration can be performed in the same manner as in the first embodiment.

The administration of the polypeptide to an analyte may be local administration or systemic administration. A path for administration may be determined appropriately in accordance with, for example, the conditions of an analyte, and it may be, for example, intravenous, intraarterial, intradermal, and intraabdominal injection or infusion. The administration amount (dosage) of the polypeptide is not particularly limited and the polypeptide may be administered in an amount sufficient for obtaining a desired contrast for imaging, for example, not more than 1 μg. The polypeptide of the present invention preferably is administered together with medicinal additives such as a carrier. The medicinal additives are as described above. The time period from the administration to the determination may be decided appropriately in accordance with, for example, a time that it takes for the polypeptide to be bonded to pancreatic β-cells, the type of the polypeptide, and the decomposition time of the polypeptide.

The method for imaging according to the second embodiment may include determining the state of pancreatic islets or pancreatic β-cells based on the obtained image using the polypeptide of the present invention. Determining the state of pancreatic islets or pancreatic β-cells includes, for example, determining the presence/absence of pancreatic islets or pancreatic β-cells and determining an increase/decrease in the amount of pancreatic islets by analyzing an image obtained as a result of the imaging of pancreatic β-cells.

Still another aspect of the present invention relates to a method for determining an amount of pancreatic islets. The method includes detecting a radioactivity signal of the polypeptide of the present invention from an analyte to which the polypeptide has been administered, and calculating an amount of the pancreatic islets from the detected signal of the polypeptide. With the method for determining an amount of pancreatic islets according to the present invention, imaging of pancreatic β-cells, and preferably imaging of GLP-1R of pancreatic β-cells can be performed because the polypeptide of the present invention is used. And from these results, an amount of pancreatic islets can be determined. The method for determining an amount of pancreatic islets according to the present invention preferably includes detecting a radioactivity signal of the polypeptide of the present invention from an analyte to which the polypeptide has been administered in advance, and calculating an amount of the pancreatic islets from the detected signal of the polypeptide.

The calculation of the amount of pancreatic islets can be performed by, for example, analyzing the detected signal amount, and an image obtained by reconfiguration of the signal. Further, the quantification of a subject of the imaging from results of the imaging can be performed easily by any person skilled in the art, using a calibration curve, an appropriate program, or the like. The subject of imaging is, for example, pancreatic islets, and preferably pancreatic β-cells. The method for determining an amount of pancreatic islets according to the present invention preferably is a method for determining an amount of pancreatic β-cells in terms of the application of the same to the examination and diagnosis.

The method for determining an amount of pancreatic islets according to the present invention further may include presenting the calculated amount of pancreatic islets. Presenting the calculated amount of pancreatic islets includes, for example, storing the calculated amount of pancreatic islets or outputting the same to the outside. Outputting the same to the outside includes, for example, displaying the same on a monitor and printing the same.

[Methods for Prevention, Treatment, and Diagnosis of Diabetes]

Still another aspect of the present invention relates to a method for prevention, treatment, or diagnosis of diabetes. As described above, in the diabetes developing process, the amount of pancreatic islets (particularly, the amount of pancreatic β-cells) decreases prior to the occurrence of glucose tolerance abnormalities, and therefore, when functional abnormalities are detected or there are subjective symptoms, diabetes has already reached the stage where it is too difficult to be treated. With the method for imaging using the polypeptide of the present invention and/or the method for determining an amount of pancreatic islets using the same, however, a decrease in the amount of pancreatic islets and/or the amount of pancreatic β-cells can be detected at an early stage, and by extension, new methods for prevention, treatment, and diagnosis of diabetes can be created. Examples of a subject (analyte) on which prevention, treatment, and diagnosis of diabetes is carried out include humans and/or mammals other than humans.

A method for diagnosis of diabetes according to the present invention includes: imaging of pancreatic β-cells with use of the polypeptide of the present invention; and determining the state of the pancreatic islets based on the obtained image of the pancreatic islets and/or the obtained amount of the pancreatic islets; and further may include performing diagnosis of diabetes based on the determination results. The determination of the state of the pancreatic islets includes, for example, determining an increase/decrease or a change in the amount of the pancreatic islets by comparing the obtained image of the pancreatic islets with an image of pancreatic islets as a reference, or comparing the obtained amount of the pancreatic islets with an amount of pancreatic islets as a reference. Further, the determination of the state of the pancreatic islets may be carried out using an information processing device. When it is determined that the amount of the pancreatic islets has decreased, preferably this information is presented, and when it is determined that the amount of the pancreatic islets has increased or has been maintained, preferably this information is presented. The diagnosis of diabetes on the basis of the determination results includes, for example, determining the risk of development of diabetes, judging it to be diabetes, and determining a degree of development of diabetes.

A method for treatment of diabetes of the present invention includes, in addition to imaging of pancreatic islets with use of the polypeptide of the present invention and performing diagnosis of diabetes based on the results of the imaging, treating diabetes on the basis of the diagnosis. The imaging of pancreatic islets and the diagnosis of diabetes can be performed in the same manner as those in the method for diagnosis of diabetes according to the present invention. The method for treatment of diabetes may include evaluating the effects of treatment such as medication and diet performed on a subject, focusing on changes in an amount of pancreatic islets.

A method for prevention of diabetes of the present invention includes imaging of pancreatic islets with use of the polypeptide of the present invention, and determining the state of the pancreatic islets based on the results of the imaging so as to determine the risk of development of diabetes. The method for prevention of diabetes of the present invention may include, for example, regularly determining the amount of the pancreatic islets, and checking the presence/absence of a tendency of a decrease in the amount of the pancreatic islets.

Another preferred aspect of the present invention relates to a method for ultra-early diagnosis of diabetes. The method for ultra-early diagnosis of diabetes of the present invention may include, for example, imaging pancreatic islets and/or determining an amount of pancreatic islets in comprehensive or ordinary medical examination by the method of the present invention, and determining the state of the pancreatic islets on the basis of the obtained image of the pancreatic islets and/or the determined amount of the pancreatic islets. Further, the method for treatment of diabetes of the present invention may include imaging pancreatic islets and/or determining an amount of pancreatic islets by the method of the present invention, and evaluating functional recovery of the pancreatic islets on the basis of the obtained image of the pancreatic islets and/or the determined amount of the pancreatic islets.

[Other Applications]

The polypeptide of the present invention may have homology with the amino acid sequence of exendin-4 (SEQ ID NO. 9) or the amino acid sequence of exendin(9-39) (SEQ ID NO. 10). As described above, it is known that exendin-4 and exendin(9-39) are GLP-1 analogues, and they bond to GLP-1R expressed on the pancreatic β-cells. For this reason, since the polypeptide of the present invention can be bonded to GLP-1R of pancreatic β-cells, preferably specifically to GLP-1R, the polypeptide of the present invention can be used in, for example, the imaging and quantification of GLP-1R-positive cells, and diagnosis and treatment of diseases involving the expression of GLP-1R. Therefore, as with the imaging and quantification of pancreatic islets, it is possible to perform the imaging and quantification of GLP-1R-positive cells and the diagnosis and/or treatment of the diseases involving the expression of GLP-1R. The diseases involving the expression of GLP-1R include, for example, neuroendocrine tumors (NET). Examples of neuroendocrine tumors include insulinoma, small cell bronchial carcinoma, and pancreatic cancer.

Hereinafter, the present invention will be further described by way of Examples and Reference Examples. It should be noted that the present invention is, when interpreted, not limited to the following Examples.

The following abbreviations will be used herein.

Ac: acetyl group
IB: 3-iodobenzoyl group
Rink Amide MBHA Resin (trade name, manufactured by Merck & Co., Inc.): 4-(2′,4′-Dimethoxyphenyl-Fmoc-aminomethyl)-phenoxyacetamido-norleucyl-MBA
HBTU: 1-[bis(dimethylamino)methylene]-1H-benzotriazolium-3-oxide-hexafluorophosphate
HOBt: 1-hydroxybenzotriazole
DMF: dimethyl formamide
Boc: tert-butoxy carbonyl group
TFA: trifluoroacetic acid
tBu: tert-butyl group
Trt: trityl group
Pbf 2,2,4,6,7-pentamethyldihydrobenzofuran-5-sulfonyl group
Fmoc: 9-fluorenylmethyloxycarbonyl group

### PEG3: —C(O)—CH₂—(OC₂H₄)₃—NH—

TIS: triisopropylsilane
DT dodecanethiol
EDTA: ethylene diamine tetra-acetic acid
HPLC: high-performance liquid chromatography
LC: liquid chromatography
PBS: phosphate-buffered saline
HEPBS: 4-(2-hydroxyethyl)-1-piperazine ethanesulfonic acid
BSA: bovine serum albumin

### EXAMPLES

### Binding Assay

A binding assay was carried out using a polypeptide represented by the formula (11) (SEQ ID No. 11) and a polypeptide represented by the formula (12) (SEQ ID No. 12) (both of which were cold probes).

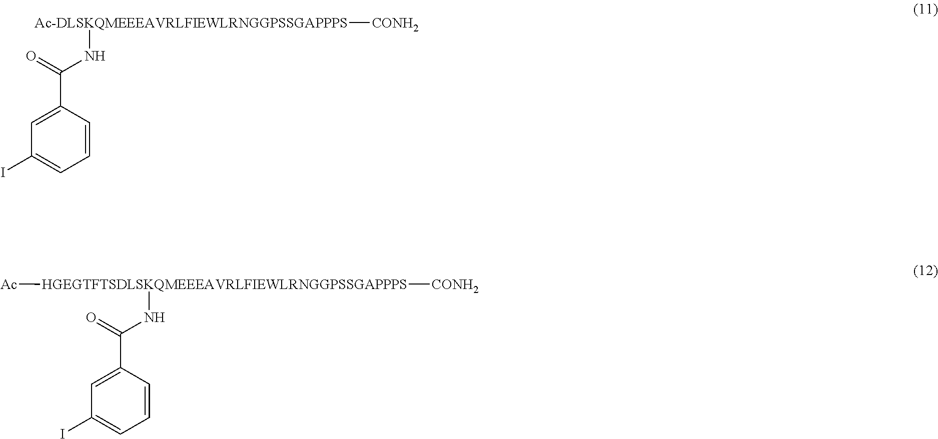

(Synthesis of Cold Probes)

### Synthesis of Polypeptide Represented by Formula (11)

The polypeptide represented by the formula (11) was prepared by following the below procedure.

First, a protected peptide resin represented by the formula (13) was synthesized. Note that protecting groups bonded to side chains other than the side chain of Lys(IB) are not recited in the formula (13).

(SEQ ID NO. 13)
Ac-DLSK(IB)QMEEEAVRLFIEWLRNGGPSSGAPPPS-Rink Amide
MBHA (13)

The protected peptide resin represented by the formula (13) was synthesized by solid phase synthesis using a peptide synthesizer (ACT90) manufactured by Advanced Chemtech. A Rink Amide MBHA Resin (0.39 mol/g, 0.25 mmol scale) was used as a starting resin carrier. Fmoc-amino acid derivatives used in a general Fmoc-peptide synthesis were used as the raw materials of the amino acids. Among the Fmoc-amino acid derivatives used, Asp(tBu), Ser(tBu), Gln(Trt), Glu(tBu), Trp(Boc), Arg(Pbf) and Asn(Trt) were used for amino acids having a functional group at their side chains. However, Fmoc-Lys(IB) was used for the lysine at position 4. The Fmoc-amino acid derivatives as the raw materials were placed in the reaction container of the peptide synthesizer to dissolve them in HBTU and HOBt as activators and DMF, and put them in a reaction vessel to let them react with each other. The obtained resin was stirred gently in piperidine-containing N-methylpyrolidone to eliminate Fmoc groups. After rinsing, condensation of the amino acid derivatives was performed as the next step, where the peptide chain was extended sequentially in accordance with the peptide sequence, and thus obtaining the protected peptide resin. After removing the Fmoc group bonded to the α-amino group of Asp at the N-terminus of the obtained protected peptide resin, the protected peptide resin was treated with acetic anhydride in accordance with a typical method so as to acetylate the α-amino group of Asp at the N-terminus, thus obtaining the protected peptide resin represented by the formula (13).

Next, the obtained protected peptide resin was treated for 2 hours at ambient temperature under typical deprotection conditions using trifluoroacetate (TFA) (TFA-TIS-H₂O-DT (95/2.5/2.5/2.5, v/v) to perform deprotection and excision of the peptide from the resin at the same time. After filtering the reaction solution to remove the carrier resin, TFA was distilled off. Ether was added to the residue to precipitate a crude product, and then the crude product was obtained by filtration.

The obtained crude peptide was fractionated and purified in a system of water-acetonitrile containing 0.1% of trifluoroacetate using a preparative high performance liquid chromatograph (HPLC) (trade name: LC-8A-2, manufactured by Shimadzu Corp., column: ODS 30×250 mm) to obtain intended peptide fractions. After distilling off the acetonitrile, the peptide fractions were made into the form of a freeze-dried powder, and thus obtaining the polypeptide represented by the formula (11) in the form of a trifluoroacetate salt.

Synthesis of Polypeptide Represented by Formula (12)

The polypeptide represented by the formula (12) was prepared by following the same procedure as that followed to obtain the polypeptide represented by the formula (11) except that a protected peptide resin represented by the formula (14) was synthesized as the protected peptide resin. Fmoc-His(Trt) was used for histidine at position 1 and Fmoc-Lys(IB) was used for the lysine at position 12. Note that protecting groups bonded to side chains other than the side chain of Lys(IB) are not recited in the formula (14).

(SEQ ID NO. 14)
Ac-HGEGTFTSDLSK(IB)QMEEEAVRLFIEWLRNGGPSSGAPPPS-
Rink Amide MBHA (14)

(Procedure of Binding Assay)

A binding assay was performed by following the below procedure. First, pancreatic islets isolated from a mouse was recovered in a 50 ml-tube and after subjecting them to centrifugation (2000 rpm, 2 minutes), they were washed once with 20 ml of cold PBS. To the washed pancreatic islets, 15 ml of trypsin-EDTA (which was prepared by adding 12 ml of PBS-containing 0.53 mM EDTA (pH 7.4 (NaOH)) to 3 ml of trypsine-EDTA (0.05%/0.53 mM)) was added. This was incubated at 37° C. for one minute while shaken, and then immediately placed on ice. Subsequently, after it was subjected to vigorous pipetting 20 times with a 10 ml pipette dropper without being foamed, cold PBS was added so that the final amount would be 30 ml. After centrifugation (3000 rpm, 2 minutes), it was washed twice with 30 ml of cold PBS. The supernatant was removed, whereby a pancreatic islet cell sample was obtained. The obtained pancreatic islet cell sample was reserved at −80° C.

The pancreatic islet cell sample was suspended in a buffer (20 mM HEPES (pH 7.4), 1 mM MgCl₂, 1 mg/ml bacitracin, 1 mg/ml BSA) so as to make 100 μL/tube. Then, 880 μL of the buffer and 10 μL of a solution containing the polypeptide represented by the formula (11) or (12) (the final concentration of the polypeptide: 0, 1×10⁻⁶ to 1×10⁻¹² M), and 10 μL of a solution containing [¹²⁵I]Bolton-Hunter labeled exendin(9-39) (prepared by adding 90 μL of a buffer to 10 μL of [¹²⁵I]Bolton-Hunter labeled exendin(9-39) (product code: NEX335, 1.85 MBq/ml=50 μCi/ml, 22.73 pmol/ml=76.57 ng/ml, manufactured by Perkin Elmer) were added thereto, which then was incubated for 60 minutes at ambient temperature. Here, the final concentration of the [¹²⁵I]Bolton-Hunter labeled exendin(9-39) was set to 0.05 μCi/tube. Next, after B/F separation by aspirating with use of an aspirator to which a pre-moistened glass fiber filter (Whatman GF/C filter) was attached, the filter was washed three times with 5 ml of ice-cold PBS. The filter was set into the tube, and the radioactivity measurement was carried out with a gamma counter.

The polypeptide represented by the formula (11) and the polypeptide represented by the formula (12) both inhibited, in a concentration-dependent manner, the binding between GLP-1R and the [¹²⁵I]Bolton-Hunter labeled exendin(9-39). IC₅₀ of the polypeptide represented by the formula (11) was 32.1 nM and IC₅₀ of the polypeptide represented by the formula (12) was 23.6 nM.

### Production Example 1

### Synthesis of Polypeptide Represented by Formula (15) (SEQ ID NO. 15)

A polypeptide represented by the formula (15) was prepared by following the below procedure.

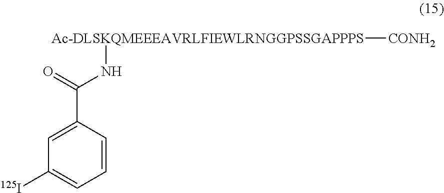

First, a protected peptide resin represented by the formula (16) was synthesized. The protected peptide resin was synthesized by following the same procedure as that followed to obtain the cold probes described above except that Fmoc-Lys(Boc) was used for the lysine at position 4. Note that protecting groups bonded to side chains other than the side chain of Lys(Boc) are not recited in the formula (16).

(SEQ ID NO. 16)
Ac-DLSK(Boc)QMEEEAVRLFIEWLRNGGPSSGAPPPS-Rink Amide
MBHA (16)

Next, the obtained protected peptide resin was treated for 2 hours at ambient temperature under typical deprotection conditions using trifluoroacetate (TFA-TIS-H₂O-DT (95/2.5/2.5/2.5, v/v) to perform deprotection and excision of the peptide from the resin at the same time. After filtering the reaction solution to remove the carrier resin, TFA was distilled off. Ether was added to the residue to precipitate a crude product, and then the crude product was obtained by filtration.

The obtained crude peptide was fractionated and purified in a system of water-acetonitrile containing 0.1% of trifluoroacetate using the preparative high performance liquid chromatograph (HPLC) (trade name: LC-8A-2, manufactured by Shimadzu Corp., column: ODS 30×250 mm) to obtain intended peptide fractions. After distilling off the acetonitrile, the peptide fractions were made into the form of a freeze-dried powder, and thus obtaining a molecular probe precursor represented by the formula (17) in the form of a trifluoroacetate salt.

(17)
(SEQ ID NO. 17)
Ac-DLSKQMEEEAVRLFIEWLRNGGPSSGAPPPS-CONH₂

The molecular probe precursor represented by the formula (17) (750 μg) was dissolved in acetonitrile and a borate buffer (pH: 7.8), and then [¹²⁵I]N-succinimidyl 3-iodobenzoate ([¹²⁵I]SIB) was added thereto. The reaction solution was adjusted to have a pH of 8.5 to 9.0 and let them react with each other for 30 minutes to bond a [¹²⁵I]3-iodobenzoyl group ([¹²⁵I]IB) to the amino group of the side chain of the lysine at position 4 to radioactively label the amino group, thus obtaining the polypeptide represented by the formula (15) as the intended product (radiochemical yield: 47.5%, radiochemical purity: >99%). Further, the time involved in the labeling was 3.5 hours. Note that the time involved in the labeling refers to the time it takes to obtain the intended labeled product (final labeled product) by letting the molecular probe precursor and the labeling compound react with each other (the same applies to the following). The time involved in the labeling in this production example includes the preparation time, the reaction time with the labeling compound, the LC purification time and the concentration time. The preparation time refers to time it takes to set into the reaction container reaction regents used for the labeling reaction, such as the labeling compound, the molecular probe precursor and the pH adjuster (the same applies to the following).

### Comparative Production Example 1

Radioactive labeling was performed in the same manner as in Production Example 1 except that a molecular probe precursor represented by the formula (18) was used in place of the molecular probe precursor represented by the formula (17) and DMF and piperidine were added to carry out a deprotection reaction after the radioactive labeling achieved by the reaction with [¹²⁵I]SIB, thus preparing a polypeptide represented by the formula (19) (SEQ ID NO. 19) (radiochemical yield: 18.4%, radiochemical purity: 96.4%). The time involved in the labeling was 5.5 hours. The time involved in the labeling in this comparative production example includes the preparation time, the reaction time with the labeling compound, the deprotection reaction time, the LC purification time and the concentration time.

(SEQ ID NO. 18)
Fmoc-DLSKQMEEEAVRLFIEWLK(Fmoc)NGGPSSGAPPPS-CONH₂
(18)

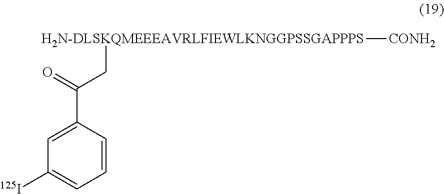

As Production Example 1 and Comparative Production Example 1 show, as a result of performing radioactive labeling with use of the molecular probe precursor represented by the formula (17) in which Lys at position 19 was replaced with Arg, the yields of the radioactively labeled polypeptide improved by 2.5 times or more and the labeled product of high purity could be obtained in comparison with the case of using the molecular probe precursor represented by the formula (18) in which Lys at position 19 was not replaced with Arg. Moreover, the time involved in the labeling could be reduced. Therefore, with the molecular probe precursor of the present invention, a radioactively labeled polypeptide of high purity can be produced efficiently.

### Production Example 2

### Synthesis of Polypeptide Represented by Formula (20) (SEQ ID NO. 20)

A polypeptide represented by the formula (20) was prepared by following the below procedure.

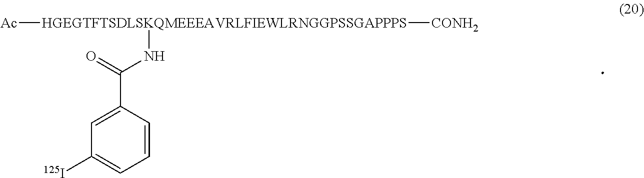

The polypeptide represented by the formula (20) (radiochemical yield: 37.0%, radiochemical purity: 99%) was prepared in the same manner as in Production Example 1 except that a molecular probe precursor represented by the formula (21) (820 μg) was used in place of the molecular probe precursor represented by the formula (17). The time involved in the labeling was 2.5 hours. The time involved in the labeling in this production example includes the preparation time, the reaction time with the labeling compound, the LC purification time and the concentration time.

(SEQ ID NO. 21)
Ac-HGEGTFTSDLSKQMEEEAVRLFIEWLRNGGPSSGAPPPS-CONH₂
(21)

### Comparative Production Example 2

Radioactive labeling was performed in the same manner as in Production Example 2 except that a molecular probe precursor represented by the formula (22) was used in place of the molecular probe precursor represented by the formula (21) and DMF and piperidine were added to carry out a deprotection reaction after the radioactive labeling achieved by the reaction with [¹²⁵I]SIB, thus preparing a polypeptide represented by the formula (23) (SEQ ID NO. 23) (radiochemical yield: 18.4%, radiochemical purity: 97.2%). The time involved in the labeling was 4.5 hours. The time involved in the labeling in this comparative production example includes the preparation time, the reaction time with the labeling compound, the deprotection reaction time, the LC purification time and the concentration time.

(SEQ ID NO. 22)
Fmoc-HGEGTFTSDLSKQMEEEAVRLFIEWLK(Fmoc)
NGGPSSGAPPPS-CONH₂ (22)

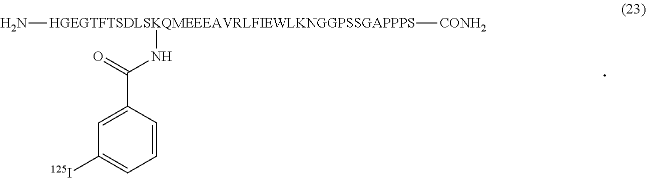

As Production Example 2 and Comparative Production Example 2 show, as a result of performing radioactive labeling with use of the molecular probe precursor represented by the formula (21) in which Lys at position 27 was replaced with Arg, the yields of the radioactively labeled polypeptide improved by twice or more and the labeled product of high purity could be obtained in comparison with the case of using the molecular probe precursor represented by the formula (22) in which Lys at position 27 was not replaced with Arg. Moreover, the time involved in the labeling could be reduced. Therefore, with the molecular probe precursor of the present invention, a radioactively labeled polypeptide of high purity can be produced efficiently.

### Example 1

A biodistribution experiment and a two-dimensional imaging analysis of mice were performed using the polypeptide represented by the formula (15) (hereinafter referred also to as “the polypeptide of Example 1”).

[Biodistribution]

The polypeptide of Example 1 (0.75 μCi) was administered to unanesthetized 6-week-old ddY mice (male, weight: 30 g) by intravenous injection (through the tail vein). After 5 minutes, 15 minutes, 30 minutes, 60 minutes, and 120 minutes from the administration, organs were dissected out of the mice (n=5). The weight and radioactivity of each organ were determined, and an accumulation amount (% dose/g) of the polypeptide of Example 1 was calculated from the radioactivity per unit weight. The exemplary results are shown in Table 1 and FIG. 1. FIG. 1 is a graph showing, by way of example, how the accumulation of the polypeptide of Example 1 in each organ changed over time. Table 2 provides the ratio of pancreas/liver (accumulation amount in pancreas/accumulation amount in liver), the ratio of pancreas/kidney (accumulation amount in pancreas/accumulation amount in kidney), and the ratio of pancreas/blood (accumulation amount in pancreas/accumulation amount in blood) based on the accumulation amount of the polypeptide of Example 1 in each organ.

TABLE 1

| | Time after administration | | | | |
|---|---|---|---|---|---|
| | 5 min | 15 min | 30 min | 60 min | 120 min |
| Pancreas | 17.01 | 17.64 | 18.88 | 12.91 | 7.69 |
| | (4.04) | (2.77) | (4.15) | (4.03) | (0.97) |
| Blood | 8.04 | 6.05 | 4.80 | 3.45 | 1.95 |
| | (0.76) | (0.22) | (0.28) | (0.45) | (0.18) |
| Heart | 4.10 | 2.94 | 2.33 | 1.78 | 0.98 |
| | (0.53) | (0.17) | (0.26) | (0.19) | (0.11) |
| Lung | 41.76 | 41.53 | 31.87 | 22.29 | 17.50 |
| | (11.22) | (4.39) | (3.57) | (5.78) | (6.19) |
| Stomach | 2.88 | 2.82 | 6.31 | 10.54 | 6.64 |
| | (0.87) | (1.16) | (5.63) | (6.69) | (2.10) |
| Intestine | 2.06 | 2.40 | 5.89 | 6.71 | 5.83 |
| | (0.45) | (0.37) | (6.03) | (1.83) | (1.14) |
| Liver | 33.51 | 23.19 | 17.73 | 14.09 | 8.79 |
| | (5.11) | (2.21) | (3.37) | (0.35) | (0.92) |
| Spleen | 3.57 | 2.67 | 2.84 | 1.80 | 0.86 |
| | (0.43) | (0.27) | (1.10) | (0.18) | (0.12) |
| Kidney | 18.32 | 16.68 | 15.98 | 12.26 | 7.39 |
| | (0.88) | (2.60) | (4.40) | (2.21) | (1.45) |
| Thyroid | 3.44 | 2.33 | 2.40 | 1.71 | 1.30 |
| gland | (0.70) | (0.54) | (1.73) | (0.67) | (0.35) |

Each numerical value indicates an average (SD) of 5 mice.

TABLE 2

| | Time after administration | | | | |
|---|---|---|---|---|---|
| | 5 min | 15 min | 30 min | 60 min | 120 min |
| Ratio of | 0.51 | 0.76 | 1.06 | 0.92 | 0.88 |
| pancreas/liver | (0.07) | (0.08) | (0.11) | (0.29) | (0.13) |
| Ratio of | 0.92 | 1.08 | 1.29 | 1.09 | 1.06 |
| pancreas/kidney | (0.18) | (0.24) | (0.51) | (0.43) | (0.18) |
| Ratio of | 2.13 | 2.92 | 3.94 | 3.89 | 3.98 |
| pancreas/blood | (0.51) | (0.49) | (0.92) | (1.59) | (0.72) |

As can be seen from Table 1 and FIG. 1, the accumulation of the polypeptide of Example 1 in the pancreas reached a level exceeding 15% dose/g at an early stage after the administration, and that level was maintained in the time period between the administration and 30 minutes after the administration. Further, no significant change was seen in the accumulation of the polypeptide of Example 1 in the thyroid gland. This suggests that the polypeptide of Example 1 was not subjected to deiodization metabolism in vivo.

The ratio of pancreas/liver in the accumulation amount of the polypeptide of Example 1 was greater than 1 in the time period after 30 minutes from the administration, and the ratio of pancreas/kidney was about 1 or greater in all of the time periods, as shown in Table 2.

[Two-Dimensional Imaging Analysis]

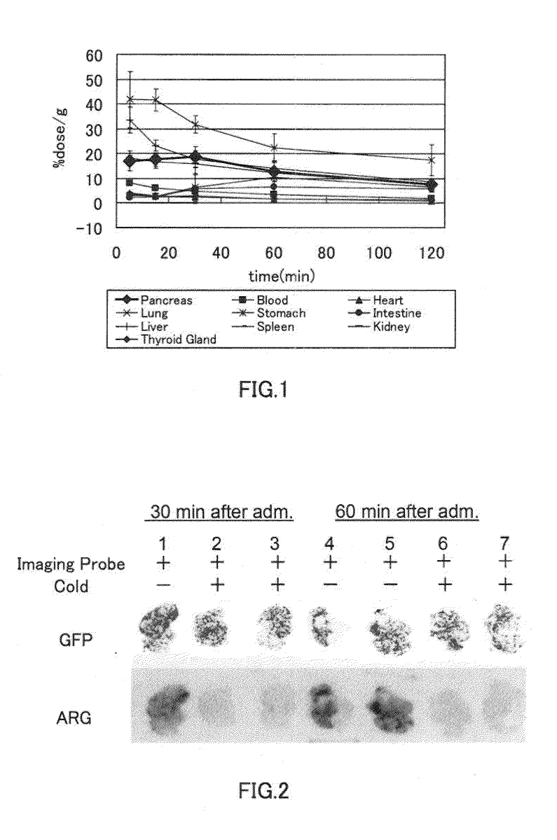

The polypeptide of Example 1 (5 μCi/100 μl) was administered to an unanesthetized MIP-GFP mouse (male, weight: 20 g) by intravenous injection, and after 30 minutes and 60 minutes from the administration, the pancreas was dissected out of the mouse (n=1). Sections were cut out of the dissected pancreas, and each section was placed on a slide glass, covered with a cover glass. The fluorescence and radioactivity (autoradiography) of each section were determined using an image analyzer (trade name: Typhoon 9410, manufactured by GE Health Care Inc.) (exposure time: 18 hours). The exemplary results are shown in lanes 1, 4 and 5 of FIG. 2.

Further, non-labeled exendin(9-39) (cold probe, SEQ ID NO. 10) was administered preliminarily to unanesthetized MIP-GFP mice (male, weight: 20 g) by intravenous injection (50 μg/100 μl). After 30 minutes from the preliminary administration, the polypeptide of Example 1 (5 μCi/100 μl) was administered to the mice by intravenous injection, and after 30 minutes and 60 minutes from the administration of the polypeptide, the pancreases were dissected out of the mice (n=2). Sections were cut out of the dissected pancreases, and the fluorescence and radioactivity of each section were determined in the same manner as above. The exemplary results are shown in lanes 2, 3, 6 and 7 of FIG. 2.

FIG. 2 illustrates exemplary results of the imaging analysis of the pancreas sections of the MIP-GFP mice to which the polypeptide of Example 1 was administered. The images on the upper row show a fluorescence signal and those on the lower row show a radioactivity signal of the polypeptide represented by the formula (15).

As shown in FIG. 2, fluorescence GFP and radioactivity signals from the pancreas sections of the MIP-GFP mice were each detected by the image analyzer. The localization of the radioactivity signal of the polypeptide of Example 1 was substantially consistent with that of the GFP signal. From this, it was confirmed that the polypeptide of Example 1 accumulated specifically in the pancreatic β-cells. Further, in the case of blocking the receptors by the preliminary administration of the cold probe the radioactivity signal of the polypeptide of Example 1 was hardly detected. This suggests that the polypeptide of Example 1 was accumulated specifically in GLP-1R of the pancreatic β-cells.

Here, ¹²⁵I, ¹²³I, and ¹³¹I were each a γ-ray emitting nuclide. Still further, ¹²⁵I and ¹²³I have the same numbers of nuclear spins. In view of these, it can be presumed that even if the polypeptide of Example 1 is modified to replace its radioactive iodine atom with a [¹²³I] iodine atom or [¹³¹I] iodine atom, it will still exhibit behaviors substantially identical to those of the polypeptide of Example 1. Further, it also can be presumed that even if the polypeptide of Example 1 is modified to replace its radioactive iodine atom with a [¹²⁴I] iodine atom, it will still exhibit behaviors substantially identical to those of the polypeptide of Example 1. This suggests that even if the polypeptide of Example 1 is modified to replace its [¹²⁵I] iodine atom with a [^{123/124/131}I] iodine atom, noninvasive three-dimensional imaging of GLP-1R of pancreatic β-cells, and preferably the quantification of GLP-1R of pancreatic β-cells can be carried out by SPECT, PET, or the like.

### Example 2

A biodistribution experiment and a two-dimensional imaging analysis of mice were performed using the polypeptide represented by the formula (20) (hereinafter referred also to as “the polypeptide of Example 2”).

[Biodistribution]

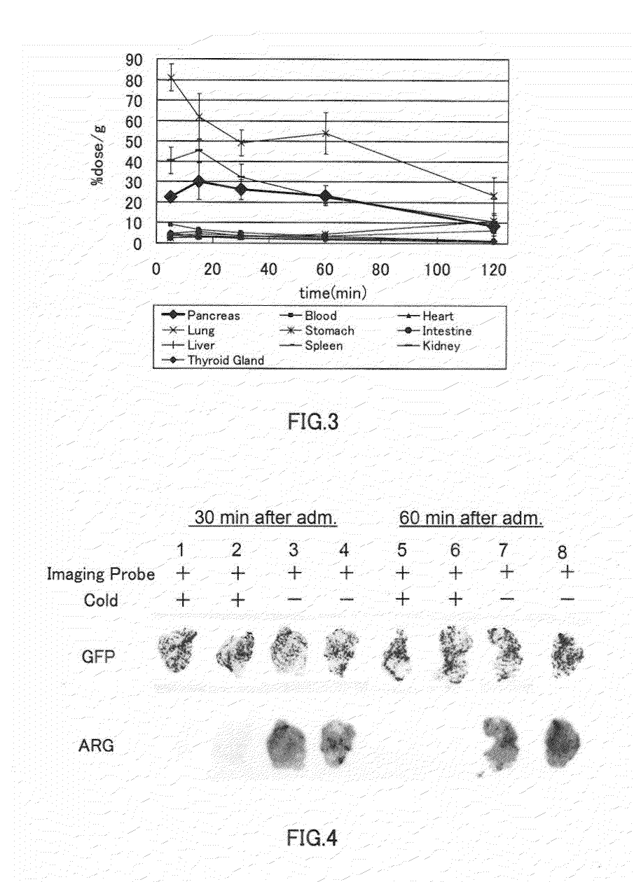

The polypeptide of Example 2 (0.51 μCi) was administered to unanesthetized 6-week-old ddY mice (male, weight: 30 g) by intravenous injection (through the tail vein). After 5 minutes, 15 minutes, 30 minutes, 60 minutes, and 120 minutes from the administration, organs were dissected out of the mice (n=5). The weight and radioactivity of each organ were determined, and an accumulation amount (% dose/g) of the polypeptide of Example 2 was calculated from the radioactivity per unit weight. The exemplary results are shown in Table 3 and FIG. 3. FIG. 3 is a graph showing, by way of example, how the accumulation of the polypeptide of Example 2 in each organ changed over time. Table 4 provides the ratio of pancreas/liver, the ratio of pancreas/kidney, and the ratio of pancreas/blood based on the accumulation amount of the polypeptide of Example 2 in each organ.

TABLE 3

| | Time after administration | | | | |
|---|---|---|---|---|---|
| | 5 min | 15 min | 30 min | 60 min | 120 min |
| Pancreas | 22.60 | 30.19 | 26.20 | 23.15 | 8.58 |
| | (0.60) | (8.97) | (4.97) | (4.91) | (3.45) |
| Blood | 9.31 | 6.50 | 4.98 | 3.56 | 1.40 |
| | (0.93) | (0.55) | (0.40) | (0.72) | (0.20) |
| Heart | 4.83 | 3.35 | 2.48 | 1.83 | 0.52 |
| | (0.66) | (0.44) | (0.12) | (0.28) | (0.31) |
| Lung | 80.97 | 61.95 | 49.18 | 53.77 | 23.51 |
| | (6.51) | (11.29) | (6.26) | (10.15) | (8.77) |
| Stomach | 2.67 | 3.43 | 3.10 | 4.37 | 11.08 |
| | (0.56) | (1.55) | (0.50) | (0.70) | (10.71) |
| Intestine | 2.46 | 2.71 | 3.02 | 3.47 | 6.20 |
| | (0.50) | (0.48) | (0.41) | (0.74) | (2.51) |
| Liver | 3.87 | 4.30 | 3.67 | 2.77 | 1.28 |
| | (0.17) | (0.35) | (0.61) | (0.68) | (0.17) |
| Spleen | 3.71 | 3.24 | 2.17 | 1.66 | 0.96 |
| | (0.77) | (0.15) | (0.18) | (0.39) | (0.25) |
| Kidney | 40.46 | 45.26 | 32.26 | 22.22 | 10.90 |
| | (6.49) | (5.76) | (6.36) | (2.89) | (2.94) |
| Thyroid | 4.74 | 5.64 | 3.52 | 2.16 | 1.24 |
| gland | (0.86) | (0.91) | (0.31) | (0.19) | (0.49) |

Each numerical value indicates an average (SD) of 5 mice.

TABLE 4

| | Time after administration | | | | |
|---|---|---|---|---|---|
| | 5 min | 15 min | 30 min | 60 min | 120 min |
| Ratio of | 5.85 | 7.06 | 7.14 | 8.66 | 6.61 |
| pancreas/liver | (0.22) | (2.17) | (0.80) | (2.51) | (2.16) |
| Ratio of | 0.57 | 0.69 | 0.84 | 1.05 | 0.79 |
| pancreas/kidney | (0.10) | (0.26) | (0.20) | (0.21) | (0.23) |
| Ratio of | 2.44 | 4.73 | 5.23 | 6.70 | 6.01 |
| pancreas/blood | (0.24) | (1.61) | (0.62) | (1.93) | (1.80) |

As can be seen from Table 3 and FIG. 3, the accumulation of the polypeptide of Example 2 in the pancreas reached a level exceeding 22% dose/g at an early stage after the administration, and the accumulation was maintained at a high level thereafter. Further, no significant change was seen in the accumulation of the polypeptide of Example 2 in the thyroid gland. This suggests that the polypeptide of Example 2 was not subjected to deiodization metabolism in vivo.

As shown in Table 4, the ratio of pancreas/liver in the accumulation amount of the polypeptide of Example 2 was greater than 5 at an early stage after the administration. Further, the ratio of pancreas/blood in the accumulation amount of the polypeptide of Example 2 was greater than 2 at an early stage after the administration, indicating a favorable blood clearance. In this way, the results suggest that clear images of pancreatic β-cells, and preferably clear images of GLP1-R of pancreatic β-cells can be obtained when imaging is performed using the polypeptide of Example 2 because the polypeptide of Example 2 accumulates highly in the pancreas while having a high pancreas/liver ratio and has an excellent blood clearance.

[Two-Dimensional Imaging Analysis]

The fluorescence and radioactivity were measured by following the same procedure as in Example 1 except that the polypeptide of Example 2 was used, the administration amount was changed to 2.7 μCi/100 μl and the exposure time was changed to 19 hours. The results are shown in FIG. 4.

FIG. 4 illustrates exemplary results of the imaging analysis of pancreas sections of MIP-GFP mice to which the polypeptide of Example 2 was administered. The images on the upper row show a fluorescence signal and those on the lower row show a radioactivity signal of the polypeptide represented by the formula (20). In FIG. 4, lanes 1 to 4 show the results of the pancreas sections dissected out from the mice after 30 minutes from the administration of the polypeptide and lanes 5 to 8 show the results of the pancreas sections dissected out from the mice after 60 minutes from the administration of the polypeptide. Further, lanes 1, 2, 5 and 6 show the results of the mouse to which the cold prove was not administered preliminary and lanes 3, 4, 7 and 8 show the results of the mouse to which the cold prove was administered preliminary.

As shown in FIG. 4, fluorescence GFP and radioactivity signals from the pancreas sections of the MIP-GFP mice were each detected by the image analyzer. Further, the localization of the radioactivity signal of the polypeptide of Example 2 was substantially consistent with that of the GFP signal. From this, it was confirmed that the polypeptide of Example 2 accumulated specifically in the pancreatic β-cells. Further, in the case of blocking the receptors by the preliminary administration of the cold probe the radioactivity signal of the polypeptide of Example 2 was hardly detected. This suggests that the polypeptide of Example 2 accumulated specifically in GLP-1R of the pancreatic β-cells.

### Example 3

Three-dimensional SPECT imaging was performed using a polypeptide represented by the formula (24) (SEQ ID NO. 24).

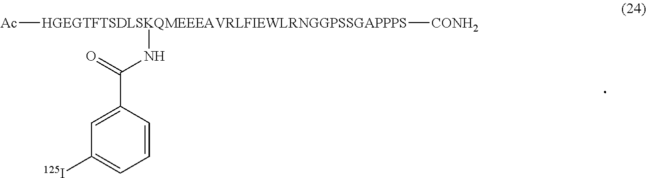

[Preparation of Polypeptide]

The polypeptide represented by the formula (24) was prepared by following the same procedure as in Production Example 2 except that [¹²³I]SIB was used in place of [¹²⁵I]SIB.

[Three-Dimensional Imaging]

The polypeptide represented by the formula (24) (480 μCi (17.8 MBq)) was administered to 6-week-old ddY mice (male, weight: about 30 g) by intravenous injection, and then after 15 minutes from the administration of the polypeptide the mice were anesthetized through enflurane inhalation. After 21 minutes from the administration of the polypeptide, the SPECT imaging was performed under the following imaging conditions with use of a gamma camera (product name: SPECT 2000H-40, manufactured by Hitachi Medical Corporation). Images obtained were reconfigured under the following reconfiguration condition.

### Imaging Conditions

Collimator: LEPH collimator
Collecting range: 360°
Step angle: 11.25°
Collecting time: 60 sec per direction

1×32 frames per 60 sec (total: 32 min)

### Reconfiguration Condition

Preprocessing filter: Butterworth filter (order: 10, cutoff frequency: 0.15)

The exemplary results are shown in FIG. 5. The images shown in FIG. 5 were taken after 21 to 53 minutes from the administration of the polypeptide. Shown in FIG. 5 are, starting from the left, a transverse view, a coronal view and a sagittal view. In the coronal view, the portion circled by a white line indicates the position of the pancreas.

As shown in FIG. 5, the position of the pancreas was confirmed noninvasively in the mice as a result of the SPECT imaging using the polypeptide represented by the formula (24). Thus, in view of that the position of the pancreas was confirmed noninvasively in a mouse that has the pancreas in a smaller size than that of a human and in which the organs are present more densely than in a human, this suggests that in a human that has the pancreas in a greater size than that of a mouse and in which the organs are present not as densely as in a mouse, the position of the pancreas and the size of the pancreas can be determined more clearly, and moreover, an amount of the polypeptide bonding to GLP-1R of pancreatic β-cells can be determined.

These results suggest that the polypeptide of the present invention allows noninvasive three-dimensional imaging of pancreatic islets in a human. For this reason, it is suggested that an amount of pancreatic β-cells (or pancreatic islets) of a human can be quantified by performing noninvasive three-dimensional imaging of GLP-1R of pancreatic β-cells using the polypeptide of the present invention.

### Production Example 3

### Synthesis of Polypeptide Represented by Formula (25) (SEQ ID NO. 25)

A polypeptide represented by the formula (25) was prepared as follows.

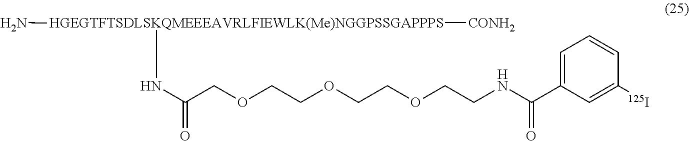

First, a molecular probe precursor represented by the formula (26) was prepared by following the same procedure as in Production Example 1 except that Fmoc-Lys(Boc-PEG3) was used for the lysine at position 12, Fmoc-Lys(Me) was used for the monomethyl lysine at position 27 and the α-amino group of His at the N-terminus was not acetylated.

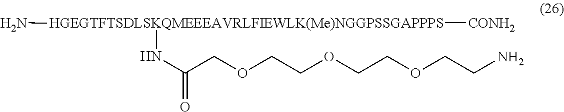

Next, the polypeptide represented by the formula (25) was prepared in the same manner as in Production Example 1 except that the molecular probe precursor represented by the formula (26) (700 μg) was used in place of the molecular probe precursor represented by the formula (21) (radiochemical yield: 33.9%, radiochemical purity: >99%). The time involved in the labeling was 2 hours. The time involved in the labeling in this production example includes the preparation time, the reaction time with the labeling compound, the LC purification time and the concentration time.

### Example 4

A biodistribution experiment on mice was performed using the polypeptide represented by the formula (25) (hereinafter referred also to as “the polypeptide of Example 4”).

[Biodistribution]

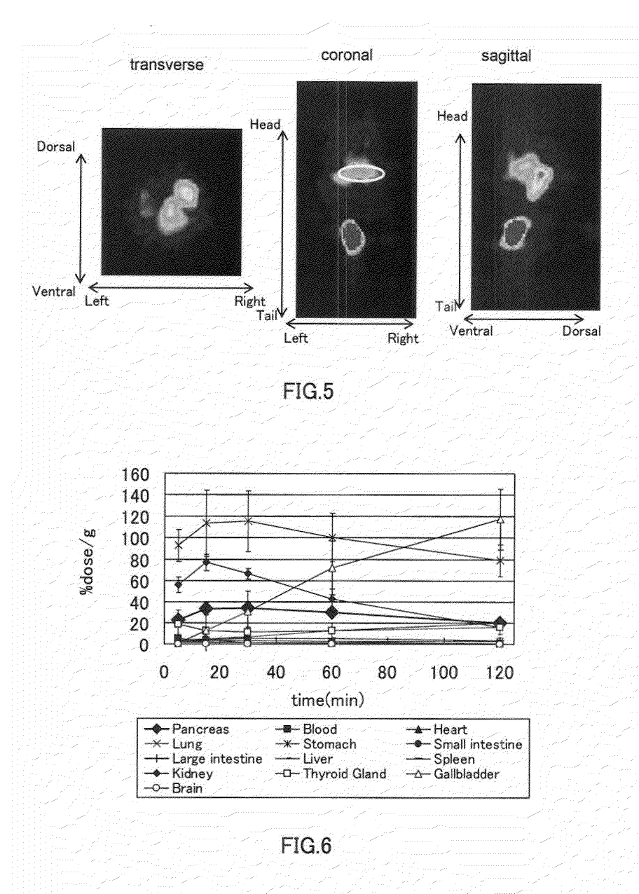

A biodistribution experiment was performed by following the same procedure as in Example 1 except that the polypeptide of Example 4 (0.61 μCi) was used in place of the polypeptide of Example 1. The exemplary results are shown in Table 5 and FIG. 6. FIG. 6 is a graph showing, by way of example, how the accumulation of the polypeptide of Example 4 in each organ changed over time. Table 6 provides the ratio of pancreas/liver, the ratio of pancreas/kidney, and the ratio of pancreas/blood based on the accumulation amount of the polypeptide of Example 4 in each organ.

TABLE 5

| | Time after administration | | | | |
|---|---|---|---|---|---|
| | 5 min | 15 min | 30 min | 60 min | 120 min |
| Pancreas | 22.79 | 32.91 | 34.05 | 29.72 | 20.03 |
| | (3.91) | (5.99) | (5.38) | (4.15) | (2.45) |
| Blood | 5.33 | 2.30 | 1.43 | 0.79 | 0.42 |
| | (0.89) | (0.31) | (0.24) | (0.16) | (0.04) |
| Heart | 2.87 | 1.79 | 1.21 | 1.07 | 0.79 |
| | (0.18) | (0.20) | (0.44) | (0.35) | (0.30) |
| Lung | 92.87 | 113.25 | 115.42 | 100.08 | 78.90 |
| | (14.88) | (30.76) | (28.22) | (22.69) | (14.93) |
| Stomach | 2.72 | 3.38 | 3.55 | 2.77 | 2.69 |
| | (0.66) | (0.71) | (0.66) | (0.74) | (0.54) |
| Small | 3.32 | 4.47 | 6.82 | 12.28 | 19.80 |
| intestine | (0.62) | (0.74) | (1.18) | (1.20) | (3.90) |
| Large | 1.32 | 1.16 | 1.09 | 1.10 | 3.25 |
| intestine | (0.21) | (0.42) | (0.16) | (0.13) | (2.63) |
| Liver | 2.30 | 3.78 | 5.08 | 5.06 | 3.02 |
| | (0.39) | (0.55) | (0.43) | (0.29) | (0.41) |
| Spleen | 2.27 | 3.85 | 1.24 | 1.11 | 0.53 |
| | (0.45) | (5.31) | (0.32) | (0.59) | (0.13) |
| Kidney | 55.86 | 76.75 | 66.27 | 42.58 | 15.98 |
| | (7.13) | (7.51) | (5.10) | (9.14) | (2.45) |
| Thyroid | 18.31 | 12.28 | 11.22 | 12.38 | 15.98 |
| Gland | (13.42) | (3.20) | (4.92) | (7.66) | (6.80) |
| Gall- | 0.66 | 12.49 | 30.75 | 71.96 | 117.23 |
| bladder | (0.61) | (20.01) | (19.19) | (25.22) | (28.32) |
| Brain | 0.18 | 0.08 | 0.06 | 0.04 | 0.03 |
| | (0.06) | (0.01) | (0.02) | (0.01) | (0.00) |

Each numerical value indicates an average (SD) of 5 mice.

TABLE 6

| | Time after administration | | | | |
|---|---|---|---|---|---|
| | 5 min | 15 min | 30 min | 60 min | 120 min |
| Ratio of | 10.34 | 8.70 | 6.74 | 5.89 | 6.66 |
| pancreas/liver | (3.49) | (0.73) | (1.20) | (0.83) | (0.40) |
| Ratio of | 0.41 | 0.43 | 0.51 | 0.72 | 1.27 |
| pancreas/kidney | (0.09) | (0.08) | (0.07) | (0.14) | (0.23) |
| Ratio of | 4.42 | 14.39 | 24.63 | 38.38 | 47.83 |
| pancreas/blood | (1.31) | (2.17) | (6.75) | (7.32) | (5.72) |

As can be seen from Table 5 and FIG. 6, the accumulation of the polypeptide of Example 4 reached a level exceeding 20% dose/g at an early stage after the administration, and this level was maintained in all of the time periods. Further, no significant change was seen in the accumulation of the polypeptide of Example 4 in the thyroid gland. This suggests that the polypeptide of Example 4 was not subjected to deiodization metabolism in vivo.

As shown in Table 6, the ratio of pancreas/liver in the accumulation amount of the polypeptide of Example 4 was greater than 5 at an early stage after the administration. Further, the ratio of pancreas/blood in the accumulation amount of the polypeptide of Example 4 was greater than 4 at an early stage after the administration, indicating a favorable blood clearance. In this way, the results suggest that clear images of pancreatic β-cells, and preferably clear images of GLP1-R of pancreatic β-cells can be obtained when imaging is performed using the polypeptide of Example 4 because the polypeptide of Example 4 accumulates highly in the pancreas while having a high pancreas/liver ratio and has an excellent blood clearance.

### Production Example 4

### Synthesis of Polypeptide Represented by Formula (27) (SEQ ID NO. 27)

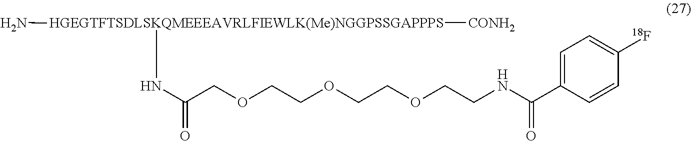

A polypeptide represented by the formula (27) was prepared in the same manner as in Production Example 3 except that the molecular probe precursor represented by the formula (26) (510 μg) was used and [¹⁸F]SFB was used in place of [¹²⁵I]SIB (radiochemical yield: 7.3%). The time involved in the labeling was 80 minutes. The time involved in the labeling in this production example includes the preparation time, the reaction time with the labeling compound, the LC purification time and the concentration time.

### Example 5

A biodistribution experiment on mice was performed using the polypeptide represented by the formula (27) (hereinafter referred also to as “the polypeptide of Example 5”).

[Biodistribution]

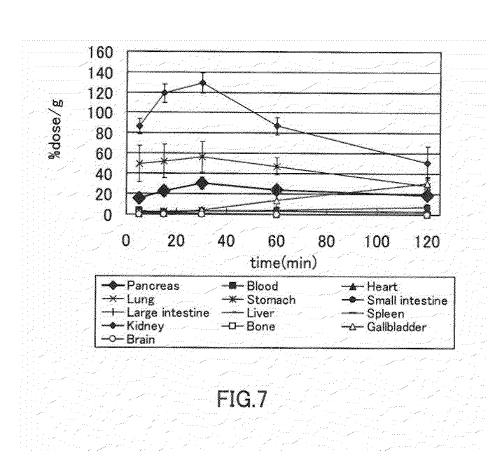

A biodistribution experiment was performed by following the same procedure as in Example 1 except that the polypeptide of Example 5 (4.2 μCi) was used in place of the polypeptide of Example 1. The exemplary results are shown in Table 7 and FIG. 7. FIG. 7 is a graph showing, by way of example, how the accumulation of the polypeptide of Example 5 in each organ changed over time. Table 8 provides the ratio of pancreas/liver, the ratio of pancreas/kidney, and the ratio of pancreas/blood based on the accumulation amount of the polypeptide of Example 5 in each organ.

TABLE 7

| | Time after administration | | | | |
|---|---|---|---|---|---|
| | 5 min | 15 min | 30 min | 60 min | 120 min |
| Pancreas | 15.88 | 22.63 | 30.18 | 24.00 | 19.17 |
| | (3.05) | (5.21) | (5.60) | (3.14) | (2.10) |
| Blood | 3.93 | 1.90 | 1.10 | 0.63 | 0.33 |
| | (0.46) | (0.10) | (0.17) | (0.03) | (0.06) |
| Heart | 2.27 | 1.28 | 0.99 | 0.69 | 0.33 |
| | (0.31) | (0.12) | (0.21) | (0.19) | (0.06) |
| Lung | 49.35 | 51.93 | 56.07 | 46.80 | 27.65 |
| | (18.15) | (16.72) | (14.89) | (8.43) | (8.43) |
| Stomach | 2.25 | 2.86 | 3.79 | 3.50 | 3.42 |
| | (0.37) | (0.69) | (0.62) | (0.63) | (0.73) |
| Small | 2.92 | 2.44 | 3.35 | 4.20 | 7.42 |
| intestine | (0.40) | (0.21) | (0.58) | (0.38) | (2.88) |
| Large | 1.23 | 0.91 | 0.89 | 0.77 | 0.77 |
| intestine | (0.24) | (0.10) | (0.16) | (0.13) | (0.21) |
| Liver | 1.67 | 1.93 | 3.09 | 3.08 | 2.15 |
| | (0.36) | (0.32) | (0.51) | (0.61) | (0.86) |
| Spleen | 1.39 | 1.04 | 0.81 | 0.43 | 0.34 |
| | (0.28) | (0.23) | (0.17) | (0.15) | (0.14) |
| Kidney | 86.48 | 118.89 | 129.29 | 86.83 | 50.42 |
| | (7.56) | (9.08) | (9.98) | (8.42) | (16.34) |
| Bone | 1.22 | 0.66 | 0.44 | 0.23 | 0.02 |
| | (0.12) | (0.14) | (0.09) | (0.13) | (0.05) |
| Gall- | 0.38 | 1.20 | 4.27 | 13.85 | 30.24 |
| bladder | (0.26) | (0.89) | (2.30) | (6.17) | (6.20) |
| Brain | 0.17 | 0.08 | 0.06 | 0.05 | 0.27 |
| | (0.02) | (0.01) | (0.01) | (0.03) | (0.45) |

Each numerical value indicates an average (SD) of 5 mice.

TABLE 8

| | Time after administration | | | | |
|---|---|---|---|---|---|
| | 5 min | 15 min | 30 min | 60 min | 120 min |
| Ratio of | 9.95 | 11.80 | 9.85 | 8.05 | 10.27 |
| pancreas/liver | (2.84) | (2.36) | (1.72) | (2.01) | (4.62) |
| Ratio of | 0.19 | 0.19 | 0.23 | 0.28 | 0.43 |
| pancreas/kidney | (0.04) | (0.05) | (0.03) | (0.05) | (0.21) |
| Ratio of | 4.12 | 11.99 | 27.37 | 38.07 | 60.91 |
| pancreas/blood | (1.05) | (3.13) | (2.53) | (5.17) | (15.01) |

As can be seen from Table 7 and FIG. 7, the accumulation of the polypeptide of Example 5 reached a level exceeding 15% dose/g at an early stage after the administration, and this level was maintained in all of the time periods. Further, no significant change was seen in the accumulation of the polypeptide of Example 5 in the bone. This suggests that the polypeptide of Example 5 was not subjected to deiodization metabolism in vivo.

As shown in Table 8, the ratio of pancreas/liver in the accumulation amount of the polypeptide of Example 5 was greater than 9 at an early stage after the administration. Further, the ratio of pancreas/blood in the accumulation amount of the polypeptide of Example 5 was greater than 4 at an early stage after the administration, indicating a favorable blood clearance. In this way, the results suggest that clear images of pancreatic β-cells, and preferably clear images of GLP1-R of pancreatic β-cells can be obtained when PET imaging is performed using the polypeptide of Example 5 because the polypeptide of Example 5 accumulates highly in the pancreas while having a high pancreas/liver ratio and has an excellent blood clearance.

As described above, the present invention is useful in, for example, the medical field, the molecule imaging field, and the field relating to diabetes.

The invention may be embodied in other forms without departing from the spirit of essential characteristics thereof. The embodiments disclosed in this application are to be considered in all respects as illustrative and not limiting. The scope of the invention is indicated by the appended claims rather than by the foregoing description, and all changes which come within the meaning and range of equivalency of the claims are intended to be embraced therein.

### Sequence Listing Free Text

SEQ ID NO. 1: exemplary amino acid sequence of molecular probe precursor of present invention

SEQ ID NO. 2: amino acid sequence of Y₁

SEQ ID NO. 3: amino acid sequence of Y₂

SEQ ID NO. 4: exemplary amino acid sequence of molecular probe precursor of present invention

SEQ ID NO. 5: exemplary amino acid sequence of molecular probe precursor of present invention

SEQ ID NO. 6: exemplary amino acid sequence of polypeptide of present invention

SEQ ID NO. 7: exemplary amino acid sequence of polypeptide of present invention

SEQ ID NO. 8: exemplary amino acid sequence of polypeptide of present invention

SEQ ID NO. 9: amino acid sequence of exendin-4

SEQ ID NO. 10: amino acid sequence of exendin(9-39)

SEQ ID NO. 11: amino acid sequence of polypeptide used in binding assay

SEQ ID NO. 12: amino acid sequence of polypeptide used in binding assay

SEQ ID NO. 13: amino acid sequence of protected peptide resin used in production of polypeptide used in binding assay.

SEQ ID NO. 14: amino acid sequence of protected peptide resin used in production of polypeptide used in binding assay

SEQ ID NO. 15: amino acid sequence of polypeptide produced in Production Example 1

SEQ ID NO. 16: amino acid sequence of protected peptide resin produced in Production Example 1

SEQ ID NO. 17: amino acid sequence of molecular probe precursor produced in Production Example 1

SEQ ID NO. 18: amino acid sequence of molecular probe precursor used in Comparative Production Example 1

SEQ ID NO. 19: amino acid sequence of polypeptide produced in Comparative Production Example 1

SEQ ID NO. 20: amino acid sequence of polypeptide produced in Production Example 2

SEQ ID NO. 21: amino acid sequence of molecular probe precursor produced in Production Example 2

SEQ ID NO. 22: amino acid sequence of molecular probe precursor used in Comparative Production Example 2

SEQ ID NO. 23: amino acid sequence of polypeptide produced in Comparative Production Example 2

SEQ ID NO. 24: amino acid sequence of polypeptide produced in Example 3 SEQ ID NO. 25: amino acid sequence of polypeptide produced in Production Example 3

SEQ ID NO. 26: amino acid sequence of molecular probe precursor produced in Production Example 3

SEQ ID NO. 27: amino acid sequence of polypeptide produced in Production Example 4

What is claimed is:

1. A method for producing a radioactively labeled polypeptide, comprising
labeling a molecular probe precursor using a labeling compound capable of labeling an amino group of a lysine or lysine derivative,
wherein the molecular probe precursor is represented by an amino acid sequence of the formula (1) and a carboxylic group at the C-terminus of the molecular probe precursor is amidated:
(1)
(SEQ ID NO. 1)
Y₁-Leu-Ser-Xaa₁₂-Gln-Met-Glu-Glu-Glu-Ala-Val-Arg-
Leu-Phe-Ile-Glu-Trp-Leu-Xaa₂₇-Asn-Gly-Y₂
where Y₁ represents an amino acid sequence represented by the formula (2) or the amino acid sequence represented by the formula (2) with 1 to 8 amino acids from the N-terminus being deleted,
Xaa₁₂ represents a lysine or lysine derivative,
Xaa₂₇ represents a basic amino acid having no functional group at its side chain that reacts with the labeling compound, or represents methyl lysine or acetylated lysine, and
Y₂ represents an amino acid sequence represented by the formula (3) or the amino acid sequence represented by the formula (3) with 1 to 9 amino acids from the C-terminus being deleted:
(2)
(SEQ ID NO. 2)
His-Gly-Glu-Gly-Thr-Phe-Thr-Ser-Asp
(3)
(SEQ ID NO. 3)
Gly-Pro-Ser-Ser-Gly-Ala-Pro-Pro-Pro-Ser.

2. The method according to claim 1, wherein the labeling compound is a compound represented by the formula (I) 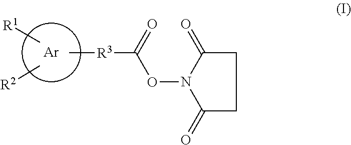
where Ar represents an aromatic hydrocarbon group or aromatic heterocyclic group, R¹ represents a substituent containing a radionuclide, R² represents a hydrogen atom or one or more substituents different from the substituent represented by R¹, and R³ represents a bond, C₁-C₆ alkylene group or C₁-C₆ oxyalkylene group.

3. The method according to claim 1, wherein Xaa₂₇ in the formula (1) represents one selected from the group consisting of arginine, monomethyl lysine, dimethyl lysine, monoacetylated lysine, norarginine, homoarginine and histidine.

4. The method according to claim 1, wherein the molecular probe precursor is represented by an amino acid sequence of the formula (4) or (5):
(4)
(SEQ ID NO. 4)
His-Gly-Glu-Gly-Thr-Phe-Thr-Ser-Asp-Leu-Ser-Xaa₁₂-Gln-Met-Glu-Glu-Glu-Ala-
Val-Arg-Leu-Phe-Ile-Glu-Trp-Leu-Xaa₂₇-Asn-Gly-Gly-Pro-Ser-Ser-Gly-Ala-Pro-
Pro-Pro-Ser
(5)
(SEQ ID NO. 5)
Asp-Leu-Ser-Xaa₁₂-Gln-Met-Glu-Glu-Glu-Ala-Val-Arg-Leu-Phe-Ile-Glu-Trp-Leu-
Xaa₂₇-Asn-Gly-Gly-Pro-Ser-Ser-Gly-Ala-Pro-Pro-Pro-Ser
where Xaa₁₂ represents a lysine or lysine derivative, and Xaa₂₇ represents a basic amino acid having no functional group at its side chain that reacts with the labeling compound, or represents methyl lysine or acetylated lysine.

5. A polypeptide represented by an amino acid sequence of the formula (6), wherein a carboxylic group at the C-terminus of the polypeptide is amidated:
(6)
(SEQ ID NO. 6)
Y₁-Leu-Ser-Xbb₁₂-Gln-Met-Glu-Glu-Glu-Ala-Val-Arg-Leu-Phe-Ile-Glu-Trp-
Leu-Xaa₂₇-Asn-Gly-Y₂
where Y₁ represents an amino acid sequence represented by the formula (2) or the amino acid sequence represented by the formula (2) with 1 to 8 amino acids from the N-terminus being deleted,
Xbb₁₂ represents a radioactively labeled lysine or lysine derivative,
Xaa₂₇ represents a basic amino acid having no functional group at its side chain that reacts with a labeling compound capable of labeling an amino group of a lysine or lysine derivative, or represents methyl lysine or acetylated lysine, and
Y₂ represents an amino acid sequence represented by the formula (3) or the amino acid sequence represented by the formula (3) with 1 to 9 amino acids from the C-terminus being deleted:
(2)
(SEQ ID NO. 2)
His-Gly-Glu-Gly-Thr-Phe-Thr-Ser-Asp
(3)
(SEQ ID NO. 3)
Gly-Pro-Ser-Ser-Gly-Ala-Pro-Pro-Pro-Ser.

6. The polypeptide according to claim 5, wherein the polypeptide is represented by an amino acid sequence of the formula (7) or (8):
(7)
(SEQ ID NO. 7)
His-Gly-Glu-Gly-Thr-Phe-Thr-Ser-Asp-Leu-Ser-Xbb₁₂-Gln-Met-Glu-Glu-Glu-Ala-
Val-Arg-Leu-Phe-Ile-Glu-Trp-Leu-Xaa₂₇-Asn-Gly-Gly-Pro-Ser-Ser-Gly-Ala-Pro-
Pro-Pro-Ser
(8)
(SEQ ID NO. 8)
Asp-Leu-Ser-Xbb₁₂-Gln-Met-Glu-Glu-Glu-Ala-Val-Arg-Leu-Phe-Ile-Glu-Trp-Leu-
Xaa₂₇-Asn-Gly-Gly-Pro-Ser-Ser-Gly-Ala-Pro-Pro-Pro-Ser
where Xbb₁₂ represents a radioactively labeled lysine or lysine derivative, and Xaa₂₇ represents a basic amino acid having no functional group at its side chain that reacts with the labeling compound, or represents methyl lysine or acetylated lysine.

7. A molecular probe precursor used in the method according to claim 1,
wherein the molecular probe precursor is represented by an amino acid sequence of the formula (1) and a carboxylic group at the C-terminus of the molecular probe precursor is amidated:
(1)
(SEQ ID No. 1)
Y₁-Leu-Ser-Xaa₁₂-Gln-Met-Glu-Glu-Glu-Ala-Val-Arg-Leu-Phe-Ile-Glu-Trp-
Leu-Xaa₂₇-Asn-Gly-Y₂
where Y₁ represents an amino acid sequence represented by the formula (2) or the amino acid sequence represented by the formula (2) with 1 to 8 amino acids from the N-terminus being deleted,
Xaa₁₂ represents a lysine or lysine derivative,
Xaa₂₇ represents a basic amino acid having no functional group at its side chain that reacts with a labeling compound capable of labeling an amino group of a lysine or lysine derivative, or represents methyl lysine or acetylated lysine, and
Y₂ represents an amino acid sequence represented by the formula (3) or the amino acid sequence of the formula (3) with 1 to 9 amino acids from the C-terminus being deleted:
(2)
(SEQ ID NO. 2)
His-Gly-Glu-Gly-Thr-Phe-Thr-Ser-Asp
(3)
(SEQ ID NO. 3)
Gly-Pro-Ser-Ser-Gly-Ala-Pro-Pro-Pro-Ser.

8. A composition for imaging comprising the polypeptide according to claim 5 or the molecular probe precursor represented by an amino acid sequence of the formula (1),
(1)
(SEQ ID No. 1)
Y₁-Leu-Ser-Xaa₁₂-Gln-Met-Glu-Glu-Glu-Ala-Val-Arg-Leu-Phe-Ile-Glu-Trp-
Leu-Xaa₂₇-Asn-Gly-Y₂
where Y₁ represents an amino acid sequence represented by the formula (2) or the amino acid sequence represented by the formula (2) with 1 to 8 amino acids from the N-terminus being deleted,
Xaa₁₂ represents a lysine or lysine derivative,
Xaa₂₇ represents a basic amino acid having no functional group at its side chain that reacts with a labeling compound capable of labeling an amino group of a lysine or lysine derivative, or represents methyl lysine or acetylated lysine, and
Y₂ represents an amino acid sequence represented by the formula (3) or the amino acid sequence of the formula (3) with 1 to 9 amino acids from the C-terminus being deleted:
(2)
(SEQ ID NO. 2)
His-Gly-Glu-Gly-Thr-Phe-Thr-Ser-Asp
(3)
(SEQ ID NO. 3)
Gly-Pro-Ser-Ser-Gly-Ala-Pro-Pro-Pro-Ser,
wherein a carboxylic group at the C-terminus of the molecular probe precursor is amidated.

9. A kit comprising the polypeptide according to claim 5 and/or the molecular probe precursor represented by an amino acid sequence of the formula (1),
(1)
(SEQ ID No. 1)
Y₁-Leu-Ser-Xaa₁₂-Gln-Met-Glu-Glu-Glu-Ala-Val-Arg-Leu-Phe-Ile-Glu-Trp-
Leu-Xaa₂₇-Asn-Gly-Y₂
where Y₁ represents an amino acid sequence represented by the formula (2) or the amino acid sequence represented by the formula (2) with 1 to 8 amino acids from the N-terminus being deleted,
Xaa₁₂ represents a lysine or lysine derivative,
Xaa₂₇ represents a basic amino acid having no functional group at its side chain that reacts with a labeling compound capable of labeling an amino group of a lysine or lysine derivative, or represents methyl lysine or acetylated lysine, and
Y₂ represents an amino acid sequence represented by the formula (3) or the amino acid sequence of the formula (3) with 1 to 9 amino acids from the C-terminus being deleted:
(2)
(SEQ ID NO. 2)
His-Gly-Glu-Gly-Thr-Phe-Thr-Ser-Asp
(3)
(SEQ ID NO. 3)
Gly-Pro-Ser-Ser-Gly-Ala-Pro-Pro-Pro-Ser,
wherein a carboxylic group at the C-terminus of the molecular probe precursor is amidated.

10. A method for imaging pancreatic β-cells, comprising
detecting a radioactivity signal of the polypeptide according to claim 5 from an analyte to which the polypeptide has been administered.

11. The method according to claim 10, further comprising reconfiguring the detected signal to convert the signal into an image, and displaying the image.

12. A method for determining an amount of pancreatic islets, the method comprising:
detecting a radioactivity signal of the polypeptide according to claim 5 from an analyte to which the polypeptide has been administered; and
calculating an amount of pancreatic islets from the detected signal of the polypeptide.

13. The method according to claim 12, further comprising presenting the calculated amount of pancreatic islets.
